(19) United States
(12) Patent Application Publication
(10) Pub. No.: US 2002/0037834 A1
(43) Pub. Date: Mar. 28, 2002

(54) Compositions and methods for enhanced sensitivity and specificity of nucleic acid synthesis

(75) Inventors: Mekbib Astatke, Germantown, MD (US); Gulilat Gebeyehu, Potomac, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Invitrogen Corporation

(21) Appl. No.: 09/948,714

(22) Filed: Sep. 10, 2001

Related U.S. Application Data
(60) Provisional application No. 60/231,330, filed on Sep. 8, 2000.

(51) Int. Cl. A61K 38/16; A61K 31/721

(52) U.S. Cl. 514/2; 514/59

(57) ABSTRACT

The present invention relates to cationic and polycationic compositions and methods for enhancing synthesis of nucleic acid molecules. In a preferred aspect, the invention relates to inhibition or control of nucleic acid synthesis, sequencing or amplification. Specifically, the present invention discloses cationic and polycationic molecules, compounds, and compositions having affinity for double-stranded and/or single-stranded nucleic acid molecules and/or single-stranded/double-stranded nucleic acid complexes (e.g., primer/template complexes, double-stranded templates, single-stranded templates or single-stranded primers) for use in such enhanced synthesis. The cationic and polycationic molecules, compounds, and compositions of the invention are capable of inhibiting nonspecific nucleic acid synthesis at ambient temperature. Thus, in a preferred aspect, the invention relates to “hot start” synthesis of nucleic acid molecules. Accordingly, the invention prevents non-specific nucleic acid synthesis at low temperatures, for example during reaction set up. The invention also relates to kits for synthesizing, amplifying, reverse transcribing or sequencing nucleic acid molecules comprising one or more of the cationic and polycationic molecules, compounds, and compositions of the invention. The invention also relates to compositions prepared for carrying out the methods of the invention and to compositions made after or during such methods. The invention also generally relates to compositions useful for inhibiting or preventing degradation of various nucleic acid molecules.

### BACKGROUND OF THE INVENTION

1. Field of the Invention

The present invention relates to a method for increasing sensitivity and specificity of nucleic acid synthesis by reducing nonspecific nucleic acid synthesis which may occur for example at ambient temperatures. The invention also relates to compositions for carrying out the methods of the invention. The methods and compositions of the present invention can be used in nucleic acid sequencing, amplification reactions, nucleic acid synthesis and cDNA synthesis.

The invention also relates to ligands (particularly cationic and polycationic molecules, compounds and compositions) which are capable of inhibiting or preventing nucleic acid synthesis, sequencing, amplification and cDNA synthesis, for example, by binding or complexing with one or more double-stranded nucleic acid molecules and/or single stranded nucleic acid molecules and/or double-stranded/single-stranded complexes. Thus the invention may inhibit or prevent nucleic acid synthesis, sequencing, amplification, and cDNA synthesis reactions by binding or interacting with nucleic acid substrates used in such reactions (e.g., primers, templates and primer/template complexes).

The invention also relates to ligands (particularly cationic and polycationic molecules, compounds and compositions) which are capable of inhibiting or preventing degradation of nucleic acid molecules during nucleic acid synthesis or preparation for nucleic acid synthesis. The ligands are capable of binding or interacting with nucleic acids, preferably single-stranded molecules or single-stranded containing molecules. Such interaction preferably prevents or inhibits degradation of the nucleic acid molecules with nucleases, particularly exonucleases and specifically single-stranded specific exonucleases. The invention also concerns kits comprising the cationic and polycationic molecules, compounds and cationic compositions of the invention.

2. Related Art

DNA polymerases catalyze the formation of DNA molecules which are complementary to all or a part of a DNA template. Upon hybridization of a primer to the single-stranded DNA template, polymerases catalyze the synthesis of DNA in the 5′ to 3′ direction, successively adding nucleotides to the 3′-hydroxyl group of the growing strand. Thus, in the presence of deoxyribonucleoside triphosphates (dNTPs) or nucleotides and a primer, a new DNA molecule, complementary to all or a part of the single stranded DNA template, can be synthesized.

Both mesophilic and thermophilic DNA polymerases are used to catalyze the formation of nucleic acids. In PCR or cycle sequencing, using thermostable rather than mesophilic polymerase is preferable due to the reduced level of non-specific DNA amplification that results from extending mis-annealed primers at less stringent annealing temperatures, e.g. ambient temperature. However, for some primer sequences and under certain experimental conditions significant amounts of synthesis of non-specific nucleic acid products reduce the sensitivity of the thermostable polymerase, requiring extensive optimization for each primer set. In addition, this problem is intensified when polymerases having high level activity at ambient temperature are employed (for example, DNA polymerase from Thermatoga neapolitana).

In examining the structure and physiology of an organism, tissue or cell, it is often desirable to determine its genetic content. The genetic framework of an organism is encoded in the double-stranded sequence of nucleotide bases in the deoxyribonucleic acid (DNA) which is contained in the somatic and germ cells of the organism. The genetic content of a particular segment of DNA, or gene, is only manifested upon production of the protein which the gene encodes. In order to produce a protein, a complementary copy of one strand of the DNA double helix (the “coding” strand) is produced by polymerase enzymes, resulting in a specific sequence of ribonucleic acid (RNA). This particular type of RNA, since it contains the genetic message from the DNA for production of a protein, is called messenger RNA (mRNA).

Within a given cell, tissue or organism, there exist many mRNA species, each encoding a separate and specific protein. This fact provides a powerful tool to investigators interested in studying genetic expression in a tissue or cell. mRNA molecules may be isolated and further manipulated by various molecular biological techniques, thereby allowing the elucidation of the full functional genetic content of a cell, tissue or organism.

A common approach to the study of gene expression is the production of complementary DNA (cDNA) clones. In this technique, the mRNA molecules from an organism are isolated from an extract of the cells or tissues of the organism. This isolation often employs chromatography matrices, such as cellulose or agarose, to which oligomers of thymidine (T) have been complexed. Since the 3′ termini on most eukaryotic mRNA molecules contain a string of adenosine (A) bases, and since A binds to T, the mRNA molecules can be rapidly purified from other molecules and substances in the tissue or cell extract. From these purified mRNA molecules, cDNA copies may be made using the enzyme reverse transcriptase (RT) or DNA polymerases having RT activity, which results in the production of single-stranded cDNA molecules. The single-stranded cDNAs may then be converted into a complete double-stranded DNA copy (i.e., a double-stranded cDNA) of the original mRNA (and thus of the original double-stranded DNA sequence, encoding this mRNA, contained in the genome of the organism) by the action of a DNA polymerase. The protein-specific double-stranded cDNAs can then be inserted into a vector, which is then introduced into a host bacterial, yeast, animal or plant cell, a process referred to as transformation or transfection. The host cells are then grown in culture media, resulting in a population of host cells containing (or in many cases, expressing) the gene of interest or portions of the gene of interest.

This entire process, from isolation of mRNA to insertion of the cDNA into a vector (e.g., plasmid, viral vector, cosmid, etc.) to growth of host cell populations containing the isolated gene or gene portions, is termed “cDNA cloning.” If cDNAs are prepared from a number of different mRNAs, the resulting set of cDNAs is called a “cDNA library,” an appropriate term since the set of cDNAs represents a “population” of genes or portions of genes comprising the functional genetic information present in the source cell, tissue or organism.

Synthesis of a cDNA molecule initiates at or near the 3′ termini of the mRNA molecules and proceeds in the 5′ to 3′ direction successively adding nucleotides to the growing strand. Priming of cDNA synthesis at the 3′ termini at the poly A tail using an oligo(dT) primer ensures that the 3′ message of the mRNAs will be represented in the cDNA molecules produced. The ability to increase sensitivity and specificity during cDNA synthesis provides more representative cDNA libraries and may increase the likelihood of the cDNA library having full-length cDNA molecules (e.g., full-length genes). Such advances would greatly improve the probability of finding full-length genes of interest.

Therefore, there is a need for a method for improving the ability of polymerases and reverse transcriptases to synthesize nucleic acid molecules. Such advances would provide for improvements in nucleic acid synthesis, sequencing, amplification and cDNA synthesis.

### SUMMARY OF THE INVENTION

The present invention satisfies the need discussed above. The present invention provides a method for inhibiting, reducing, substantially reducing or eliminating nucleic acid synthesis and/or degradation under certain conditions (preferably at ambient temperatures). In a preferred aspect, the invention prevents or inhibits nucleic acid synthesis and degradation (specifically template and primer degradation) during reaction set up and preferably before optimum reaction conditions for nucleic acid synthesis are achieved. Thus, the invention allows inhibition of polymerase and/or nuclease activities used in or present during nucleic acid synthesis. Such inhibition of DNA polymerase activities at sub-optimum conditions or during reaction set up prevents or reduces non-specific nucleic acid synthesis. Once reaction set up is complete and the optimum conditions are reached, nucleic acid synthesis can be initiated. Moreover, the invention prevents degradation of nucleic acid synthesis substrates and products and thus may provide for more efficient nucleic acid synthesis after synthesis begins.

More specifically, the invention relates to controlling nucleic acid synthesis by introducing any one or more ligands (particularly cationic or polycationic molecules, compounds or compositions) which bind to or interact with any nucleic acid molecules such as single-stranded or double-stranded nucleic acids, or double-stranded containing nucleic acid molecules including double-stranded/single-stranded complexes. Such double-stranded nucleic acid molecules may contain single-stranded regions (preferably at one or both termini), or may contain sequences or nucleotides which are not base paired with a complementary nucleic acid strand, or may be completely double-stranded. Accordingly, such cationic or polycationic molecules, compounds or compositions can bind or interact with such double-stranded nucleic acid molecules (e.g., double-stranded substrates such as a primer/template complex or a double-stranded template) and interfere with nucleic acid synthesis by preventing binding or interaction of an active polymerase or reverse transcriptase with nucleic acid synthesis substrates such as primer/template complex.

In another aspect, the invention relates to controlling nucleic acid synthesis by introducing any one or more ligands (particularly cationic or polycationic molecules, compounds or compositions) which bind to nucleic acids, particularly double-stranded, single-stranded or single-stranded containing nucleic acids. Accordingly, such cationic or polycationic molecules, compounds or compositions can bind to or interact with nucleic acid molecules (e.g., nucleic acid synthesis substrates such as single stranded primers or single stranded templates or double-stranded molecules) and interfere with nucleic acid synthesis, for example, by preventing binding or interaction or hybridization of the nucleic acid synthesis substrates (such as primer with the template to form the primer/template complex substrate used by polymerases or reverse transcriptases in synthesis reactions).

In addition, the interaction of the ligands (particularly cationic or polycationic molecules, compounds or compositions) of the invention with nucleic acid molecules, particularly single-stranded nucleic acids (e.g., single-stranded substrates such as primers and templates) prevents such molecules from being degraded by nucleases (such as exonucleases) that may be present. The cationic or polycationic molecules, compounds or compositions of the invention thus prevents degradation of substrates used in nucleic acid synthesis, amplification and sequencing reactions, but also prevents degradation of the products produced by such reactions. For example, numerous polymerases used in nucleic acid synthesis, amplification and sequencing have exonuclease activity (e.g., 3′ to 5′ and 5′ to 3′ exonuclease activity of DNA polymerases) which may degrade single-stranded nucleic acid substrates or products and adversely affect the efficiency of a nucleic acid synthesis reaction. Moreover, reaction mixtures used in synthesis, amplification and sequencing may contain added nucleases (which may be added to the reaction mixture for a particular purpose or function) or contaminating nucleases (erg., RNase's, DNase's, and exonucleases and specifically single-stranded exonucleases) which may degrade nucleic acid substrates or products in the reaction mixture. By including the cationic or polycationic molecules, compounds or compositions of the invention, it is possible to prevent or inhibit degradation of the nucleic acid molecules or substrates before, during or after nucleic acid synthesis, amplification and sequencing.

The invention thus relates to ligands which bind to (preferably by non-cationic binding) or interact with nucleic acid molecules and preferably form ligand/nucleic acid complexes. Nucleic acid ligands of the invention (which can be called “inhibitory ligands” or “nucleic acid ligands”) can be any molecule or compound (including chemical compounds and polymers) which has a charge profile such that it binds or interacts with any nucleic acid molecule such as double-stranded nucleic acid molecules and/or single-stranded nucleic acid molecules and/or single-stranded/double-stranded nucleic acid complexes, preferably condensing the structure of the nucleic acid. Preferred ligands include natural and synthetic compounds, peptides, polypeptides, proteins, lipids, lipoproteins, and the like. In general, ligands of the invention include any cationic or polycationic molecule, compound or composition. Natural cationic molecules include histones, protamine, spermine, spermidine, and high mobility group proteins (Biochim Biophys Acta 1988, 950, 221-228; Science 1989, 243, 375-378; Proc Natl Acad Sci USA 1991, 88, 4255-4259). Synthetic cationic molecules include organic molecules or polymers such as DEAE-dextran, polybrene, polylysine, polyhistidine, cationic polypeptides, macromolecules with a cationic core (for review please see Cotten, M and Wagner, E 1993, Curr. Opin. Biotechnol. 4, 705-710; Bioconjugate Chem. 4, 372-379), amphiphilic aggregates (Behr, J. P., 1994, Bioconjugate Chem. 5, 382-389), polyamidoamine cascade polymers or dendrimers, lipopolyamines, and polyethylenimine (Boussif et al., 1995, Proc. Natl. Acad. Sci. USA 92, 7297-7301). Also included is a nonlipid, nonpeptide polycationic polymer, a synthetic polyamino polymer with a glucose backbone described in Goldman, C. K. et al., 1997 (Nature Biotech 15, 462-466). Other compositions include cationic lipids, or cationic liposome formulations such as “Transfectam™” (Promega), “DOTAP™” (Roche), “FUGENE 6™” (Roche), “X-treme GENE Q2™” (Roche), “GeneJammer™” (Stratagene), “GenePorter™” (Gene Therapy Systems), “Effectene™” (Quiagen), “Superfect™” (Quiagen), “LIPOFECTIN®” (Invitrogen Corporation, Life Technologies Division), “LIPOFECTACE™” (Invitrogen Corporation, Life Technologies Division), “LIPOFECTAMINE™” (Invitrogen Corporation, Life Technologies Division), “LIPOFECTAMINE 2000™” (Invitrogen Corporation, Life Technologies Division), “CELLFECTIN®” (Invitrogen Corporation, Life Technologies Division), “DMRIE-C™” (Invitrogen Corporation, Life Technologies Division), and others described in U.S. Pat. Nos. 4,812,449, 4,891,355, 5,171,678, 5,186,923, 5,208,036, 5,264,618, 5,277,897, 5,279,833, 5,283,185, 5,334,761, 4,897,355, 5,459,127, 5,545,412, 5,650,096, 5,667,774, 5,674,908, 5,705,385, 5,719,131, 5,736,392, 5,744,335, 5,783,565, 5,830,430, 5,840,710, 5,854,224, 5,869,606, 5,906,922, 5,935,936, 5,948,925, 5,948,767, WO 97/42819, WO 98/02190, WO 98/17373, WO 98/19709, WO 99/29712, WO 98/40499, WO 98/40502, WO 98/42819, EP 0394111, EP 0846680, and FR 1,567,214. U.S. Pat. No. 5,861,397 describes amphiphilic cationic lipids, and U.S. Pat. No. 5,670,347 describes a synthetic polypeptide which interacts with nucleic acids. In general, DNA condensing agents and transfection agents also be used in accordance with the invention. In one aspect, the ligands of the invention are not nucleic acid molecules and/or are not enzymes, which are capable of binding nucleic acid molecules.

In a another preferred aspect, the ligands (e.g., cationic or polycationic molecules, compounds) and compositions of the present invention are capable of binding (preferably by non-covalent binding) or forming complexes with one or more nucleic acid molecules and particularly one or more nucleic acid synthesis substrates under certain conditions and can dissociate from the nucleic acids when the conditions are changed. Conditions include varying temperature, ionic strength and pH of mixture. Thus, the cationic or polycationic molecules, compounds and compositions are preferably introduced into the reaction mixture where it competitively binds to or interacts with the substrate(s) (e.g., primer/template complexes, double stranded molecules and/or single-stranded molecules such as single-stranded primers and single stranded templates), thereby inhibiting nucleic acid synthesis in the presence of one or more enzymes having polymerase or reverse transcriptase activity under particular reaction conditions. The cationic or polycationic molecules, compounds and compositions of the invention also have the ability to interact or bind with the synthesized products and/or substrates of the reaction mixture, thereby preventing degradation of the products or substrates with nucleases which may be present in the reaction mixture, resulting in an increase in nucleic acid synthesis products.

Thus, in a preferred aspect, one or more cationic or polycationic molecules, compounds and compositions of the invention are capable of binding one or more nucleic acid substrates, and are capable of preventing synthesis with such substrates (e.g., single-stranded templates and single-stranded primers) under certain conditions. Such synthesis is prevented, for example, by preventing interaction of the nucleic acids with active polymerases/reverse transcriptases and/or by preventing interaction of the nucleic acid molecules (such as hybridization to form primer/template complexes). Such cationic or polycationic molecules, compounds and compositions also prevent degradation of nucleic acid molecules in the reaction since they bind such molecules, preferably making them inaccessible to the action of nucleases. Thus, such cationic or polycationic molecules, compounds and compositions are preferably introduced into a reaction mixture where it competitively binds to or interacts with such nucleic acid molecules, thereby inhibiting nucleic acid synthesis and/or nucleic acid degradation in the presence of one or more enzymes having polymerase and/or nuclease activity.

The inhibition of nucleic acid synthesis or the interaction/binding by the ligands (e.g., cationic or polycationic molecules, compounds and compositions) of the invention is preferably eliminated or reduced so that nucleic acid synthesis may proceed when reaction conditions are changed, for example, when the temperature is raised. In a preferred aspect, the changed conditions affect the ability of the cationic or polycationic molecules to interact with double-stranded nucleic acid substrates and/or single-stranded nucleic acid substrates and/or single-stranded/double-stranded complexes, causing release of the substrates (e.g., dissociation of the cationic/polycationic molecules from the substrates) and/or denaturation or inactivation of the cationic or polycationic molecules making the nucleic acid molecules available as substrates for the enzyme with polymerase/reverse transcriptase activity thus allowing nucleic acid synthesis to proceed.

The invention therefore relates to a method for synthesizing one or more nucleic acid molecules, comprising (a) mixing one or more nucleic acid templates (which may be a DNA molecule such as a cDNA molecule, or an RNA molecule such as a mRNA molecule) with one or more primers, and one or more ligands (e.g., cationic or polycationic molecules, compounds and compositions) of the present invention capable of binding or interacting with one or more double-stranded and/or single-stranded nucleic acid substrates and/or single-stranded/ double-stranded complexes (e.g., substrates for nucleic acid synthesis such as templates, template/primer complexes and/or primers) and (b) incubating the mixture in the presence of one or more enzymes having nucleic acid polymerase activity and/or nuclease activity (e.g., DNA polymerases and/or reverse transcriptases and/or nucleases such as endonucleases and exonucleases) under conditions sufficient to synthesize one or more first nucleic acid molecules complementary to all or a portion of the templates. Such mixing is preferably accomplished under conditions to prevent nucleic acid synthesis and/or to allow binding of the ligands (e.g., cationic or polycationic molecules, compounds and compositions) of the invention to one or more nucleic acid synthesis substrates. In a preferred aspect, the synthesis conditions are sufficient to dissociate the ligands from the nucleic acid or denature the ligands of the invention to inhibit, reduce, substantially reduce or eliminate binding of said ligands to the nucleic acid synthesis substrates. In one embodiment of the invention, the cationic/polycationic molecules or compounds (e.g., lipid or liposomal formulations) are able to renature or regain their ability to bind nucleic acid once the incubation conditions are reestablished for such an association. Such incubation conditions may involve the use of one or more nucleotides and one or more nucleic acid synthesis buffers. Thus, preferred ligands (e.g., cationic/polycationic molecules or compounds) of the invention reversibly associate/dissociate with nucleic acid molecules depending on the conditions used. Accordingly, several cycles of synthesis can take place by varying the incubation conditions without the need to add additional cationic/polycationic compounds during the reaction. For example, cycling of a reaction at different conditions, for example during amplification (e.g., PCR), will not inactivate the cationic/polycationic molecules and thus such molecules may bind or associate with the nucleic acid synthesis substrates and synthesis products once conditions are reached which allow such interaction. Preferably, the incubation conditions are accomplished at a temperature sufficient to dissociate the cationic/polycationic molecules of the invention and/or prevent binding of the cationic/polycationic molecules to the nucleic acid synthesis substrates, but at a temperature insufficient to inactivate the polymerases and/or reverse transcriptases or other enzymes present and needed for the nucleic acid synthesis reaction. Such methods of the invention may optionally comprise one or more additional steps, such as incubating the synthesized first nucleic acid molecules under conditions sufficient to make one or more second nucleic acid molecules complementary to all or a portion of the first nucleic acid molecules. Such additional steps may also be accomplished in the presence of the ligands (e.g., cationic/polycationic molecules) of the invention as described herein. The invention also relates to nucleic acid molecules synthesized by this method.

Using the method of the present invention, the synthesized nucleic acid molecules can be used directly in other assays or procedures where the presence of the ligand/nucleic acid mixture or complex (e.g., nucleic acid/cationic or polycationic complex) is beneficial, such as for introduction of nucleic acids into hosts or host cells, or where the presence of nucleic acid/cationic or polycationic compound does not dramatically affect the final goal of the assay. Thus, the invention more specifically relates to introduction of nucleic acid molecules into one or more host or host cells comprising: (a) synthesizing one or more nucleic acid molecules in the presence of the ligands (particularly cationic or polycationic molecules or transfection agents) of the invention; and (b) introducing said synthesized nucleic acid molecules into one or more host or host cells in the presence of said ligands of the invention.

More specifically, the invention relates to a method of amplifying a DNA molecule comprising: (a) mixing a first and second primer, wherein said first primer is complementary to a sequence at or near the 3′-termini of the first strand of said DNA molecule and said second primer is complementary to a sequence at or near the 3′-termini of the second strand of said DNA molecule and one or more ligands (e.g., cationic or polycationic molecules, compounds or compositions) of the invention (e.g., a molecule with affinity to double-stranded nucleic acids and/or single-stranded nucleic acids and/or single-stranded/double-stranded complexes); (b) hybridizing said first primer to said first strand and said second primer to said second strand; (c) incubating the mixture under conditions such that a third DNA molecule complementary to all or a portion of said first strand and a fourth DNA molecule complementary to all or a portion of said second strand are synthesized; (d) denaturing said first and third strand, and said second and fourth strands; and (e) repeating steps (a) to (c) or (d) one or more times. Such mixing is preferably accomplished under conditions to prevent nucleic acid synthesis and/or to allow binding of the cationic or polycationic molecules, compounds or compositions of the invention to one or more nucleic acid synthesis substrates. In a preferred aspect, the synthesis conditions are sufficient to dissociate or denature, or reduce the ability of the cationic or polycationic molecules, compounds or compositions of the invention to inhibit, reduce, substantially reduce or eliminate binding of said cationic or polycationic molecules, compounds or compositions to the nucleic acid synthesis substrates. Preferably, the incubation conditions are accomplished at a temperature sufficient to dissociate the cationic or polycationic molecules, compounds or compositions of the invention and/or prevent binding of the cationic or polycationic molecules, compounds or compositions to the nucleic acid synthesis substrates, but at a temperature insufficient to denature or inactivate the polymerases and/or reverse transcriptases or other enzymes present and needed for the nucleic acid synthesis reaction. Such incubation conditions may include incubation in the presence of one or more polymerases, one or more nucleotides and/or one or more buffering salts. The invention also relates to nucleic acid molecules amplified by these methods. Such amplified nucleic acid molecules made accordingly to the methods of the invention may also be further manipulated or processed including introduction of the amplified nucleic acid molecules into one or more hosts or host cells. Thus the invention specifically relates to introduction of nucleic acid molecules into one or more host or host cells comprising: (a) amplifying one or more nucleic acid molecules in the presence of one or more ligands (e.g., cationic or polycationic molecules, compounds or compositions) of the invention; and (b) introducing said amplified nucleic acid molecules into one or more host or host cells in the presence of at least one of said ligands.

The invention also relates to methods for sequencing a nucleic acid molecule comprising (a) mixing a nucleic acid molecule to be sequenced with one or more primers, one or more of the ligands (e.g., cationic or polycationic molecules, compounds or compositions) of the invention, one or more nucleotides and one or more terminating agents to form a mixture; (b) incubating the mixture under conditions sufficient to synthesize a population of molecules complementary to all or a portion of the molecule to be sequenced; and (c) separating the population to determine the nucleotide sequence of all or a portion of the molecule to be sequenced. The invention more specifically relates to a method of sequencing a nucleic acid molecule, comprising: (a) mixing a cationic or polycationic molecules, compounds or compositions of the present invention (having affinity to double-stranded nucleic acids and/or single stranded nucleic acids and/or single-stranded/double-stranded complexes), one or more nucleotides, and one or more terminating agents; (b) hybridizing a primer to a first nucleic acid molecule; (c) incubating the mixture of step (b) under conditions sufficient to synthesize a random population of nucleic acid molecules complementary to said first nucleic acid molecule, wherein said synthesized molecules are shorter in length than said first molecule and wherein said synthesized molecules comprise a terminator nucleotide at their 3′ termini; and (d) separating said synthesized molecules by size so that at least a part of the nucleotide sequence of said first nucleic acid molecule can be determined. Such mixing is preferably accomplished under conditions to prevent nucleic acid synthesis and/or to allow binding of the cationic or polycationic molecules, compounds or compositions of the invention to one or more nucleic acid synthesis substrates. In a preferred aspect, the synthesis conditions and/or hybridization conditions are sufficient to dissociate or denature the cationic or polycationic molecules, compounds or compositions of the invention to inhibit, reduce, substantially reduce or eliminate binding of said cationic or polycationic molecules, compounds or compositions to the nucleic acid synthesis substrates. Preferably, the incubation conditions are accomplished at a temperature sufficient to dissociate or reduce the binding of the cationic/polycationic molecules of the invention and/or prevent binding of the cationic/polycationic molecules to the nucleic acid synthesis substrates, but at a temperature insufficient to inactivate the polymerases and/or reverse transcriptases or other enzymes present and needed for the nucleic acid synthesis reaction. Such terminator nucleotides include ddNTP, ddATP, ddGTP, ddITP or ddCTP, or modified derivatives thereof. Such incubation conditions may include incubation in the presence of one or more polymerases and/or buffering salts.

The invention also generally relates to methods of preventing or inhibiting the degradation of nucleic acid molecules in a nucleic acid synthesis reaction. Preferably, such methods are preformed during nucleic acid synthesis, cDNA synthesis, amplification or sequencing. Specifically, the methods may comprise: (a) obtaining one or more ligands (e.g., cationic/polycationic molecules) of the invention, and (b) contacting said ligands of the invention with one or more nucleic acid molecules under conditions sufficient to prevent or inhibit degradation of said nucleic acid molecules with one or more nucleases having nuclease activity. The cationic/polycationic molecules of the invention have affinity for and thus may bind or interact with nucleic acid molecules. Accordingly, the cationic/polycationic molecules of the invention are capable of binding nucleic acids and thus preventing interaction or binding of nucleases with such nucleic acid molecules. In a preferred aspect, the methods of protecting nucleic acid molecules according to the invention are accomplished during in vitro reactions, particularly those reactions used in standard molecular biology techniques (such as nucleic acid synthesis, amplification, sequencing and cDNA synthesis). The degradation protection method of the invention may further comprise the step of dissociating the cationic/polycationic molecules of the invention and/or preventing binding of the cationic/polycationic molecules of the invention to the nucleic acid molecules under particular conditions, for example, by increasing temperature, altering pH, or changing the ionic strength of the reaction mixture.

The invention also relates to the ligands (e.g., cationic/polycationic molecules) of the invention and to compositions comprising the ligands of the invention, as well as nucleic acid molecules produced by the methods of the invention, to vectors (which may be expression vectors) comprising these nucleic acid molecules, and to host cells comprising these nucleic acid molecules or vectors. The ligands (e.g., cationic/polycationic molecules, compounds or compositions) for use in the invention can be produced by well known techniques, for example, methods described in U.S. Pat. Nos. 4,812,449, 5,171,678, 5,186,923, 5,277,897, 5,208,036, 5,208,036, 5,264,618, 5,279,833, 5,334,761, 4,897,355, 5,459,127, 5,650,096, 5,744,335, 5,854,224, 5,869,606, 5,906,922, 5,674,908, and WO 98/19709. U.S. Pat. No. 5,861,397 describes production of amphiphilic cationic lipids, and U.S. Pat. No. 5,670,347 describes production of a synthetic polypeptide which interacts with nucleic acids.

The invention also relates to kits for use in synthesis, sequencing and amplification of nucleic acid molecules, comprising one or more containers containing one or more of the ligands (e.g., cationic or polycationic molecules, compounds or compositions) of the invention. These kits of the invention may optionally comprise one or more additional components selected from the group consisting of one or more nucleotides, one or more templates, one or more polymerases (e.g., thermophilic or mesophilic DNA polymerases) and/or reverse transcriptases, one or more suitable buffers, one or more primers, one or more terminating agents (such as one or more dideoxynucleotides), and instructions for carrying out the methods of the invention. The invention also relates to kits for use in the general methods of preventing or inhibiting degradation of nucleic acid molecules according to the invention. Such kits may comprise one or more containers containing one or more of the ligands (e.g., cationic or polycationic molecules, compounds or compositions) of the invention. These kits may optionally comprise one or more additional components selected from the group consisting of one or more nucleotides, one or more templates, one or more polymerases (e.g., thermophilic or mesophilic DNA polymerases) and/or reverse transcriptases, one or more nucleases, one or more suitable buffers, one or more primers, one or more terminating agents, and instructions for carrying out this method of the invention.

The invention also relates to compositions for use in synthesis, sequencing and amplification of nucleic acid molecules and to compositions made for carrying out such synthesis, sequencing and amplification reactions. The invention also relates to compositions made during or after carrying out the synthesis, sequencing and amplification reactions of the invention. Such compositions of the invention may comprise one or more of the ligands (e.g., inhibitory cationic/polycationic molecules) of the invention and may further comprise one or more components selected from the group consisting of one or more nucleotides, one or more primers, one or more templates, one or more reverse transcriptases, one or more DNA polymerases, one or more buffers, one or more buffer salts and one or more synthesized nucleic acid molecules made according to the methods of the invention. The invention also relates to the compositions for use in the methods of preventing or inhibiting degradation in nucleic acid molecules and to compositions made for carrying out such methods. The invention also relates to compositions made during or after carrying out such methods of protecting against degradation in nucleic acid molecules. Such compositions of the invention may comprise one or more of the ligands (e.g., inhibitory cationic/polycationic molecules) of the invention and may further comprise one or more components selected from the group consisting of one or more nucleotides, one or more primers, one or more templates, one or more reverse transcriptases, one or more polymerases (DNA polymerases and reverse transcriptases), one or more buffers, one or more buffering salts, and one or more nucleic acid molecules.

Other preferred embodiments of the present invention will be apparent to one of ordinary skill in light of the following drawings and description of the invention, and of the claims.

### BRIEF DESCRIPTION OF THE DRAWINGS/FIGURES

FIG. 1 shows the inhibition of a DNA polymerization reaction catalyzed by Tne DNA polymerase by LIPOFECTAMINE™. The Tne DNA polymerase used in all the measurement reported here is deficient of the 5′-3′ exo-activity due to the introduction of Asp137Ala substitution (See U.S. Pat. No. 5,948,614). P denotes the position of the DNA primer (34-mer) and F.L. is the fully extended product (60-mer). Lane Q is a control lane of the oligonucleotide substrate. Panels I, II, III, and IV indicate the polymerase reactions catalyzed by Tne at varying concentrations of LIPOFECTAMINE™. Panel I represents the reaction in the absence of LIPOFECTAMINE™; Panels II, III, and IV represent the reaction in the presence of 10 mM, 20 mM and 40 mM of LIPOFECTAMINE™, respectively. For each reaction condition the DNA substrate and the Tne DNA polymerase concentrations were maintained at about 10 mM and 70nM, respectively. The polymerase reaction was measured at ambient temperature, 37° C. and 72° C. as represented by the sub-panels of a, b, and c, respectively. For each condition the reaction was stopped at 4 minutes following the initiation of polymerization by the addition of Tne.

FIG. 2 shows the inhibition of the 3′-5′ exo-nuclease reaction catalyzed by the Tne DNA polymerase using LIPOFECTAMINE™ at ambient temperature. P denotes the position of the 34-mer DNA substrate. Lane Q is the control lane of the oligonucleotide substrate. Panels I, II, III, IV, and V indicate the 3′-5′ exo-nuclease reactions catalyzed by Tne DNA polymerase at varying concentrations of the LIPOFECTAMINE™. Panel I represents the reaction in the absence of LIPOFECTAMINE™; Panels II, III, IV and V represent reactions in the presence of 10 mM, 20 mM, 40 mM and 60 mM of LIPOFECTAMINE ™, respectively. For each reaction condition the DNA substrate and Tne DNA polymerase concentrations were maintained at about 10 nM and 70 nM, respectively. The exo-nuclease digestion of the 34-mer substrate was measured at ambient temperature, 37° C. and 72° C. as represented by the sub-panels of a, b, and c, respectively. For each reaction condition the digestion was stopped at 20 minutes following the initiation of the reaction by the addition of Tne.

### DETAILED DESCRIPTION OF THE INVENTION

Definitions

In the description that follows, a number of terms used in recombinant DNA technology are utilized extensively. In order to provide a clearer and consistent understanding of the specification and claims, including the scope to be given such terms, the following definitions are provided.

Primer. As used herein, “primer” refers to a single-stranded oligonucleotide or DNA that is extended by covalent bonding of nucleotide monomers during amplification or polymerization of a nucleic acid molecule.

Template. The term “template” as used herein refers to double-stranded or single-stranded nucleic acid molecules (RNA and/or DNA) which are to be amplified, synthesized or sequenced. In the case of a double-stranded molecules, denaturation of its strands to form a first and a second strand is preferably performed before these molecules may be amplified, synthesized or sequenced, or the double-stranded molecule may be used directly as a template. For single stranded templates, a primer, complementary to a portion of the template is hybridized under appropriate conditions and one or more polymerases may then synthesize a nucleic acid molecule complementary to all or a portion of said template. Alternatively, for double-stranded templates, one or more promoters (e.g., SP6, T7 or T3 promoters) may be used in combination with one or more polymerases to make nucleic acid molecules complementary to all or a portion of the template. The newly synthesized molecules, according to the invention, may be equal or shorter in length than the original template.

Incorporating. The term “incorporating” as used herein means becoming a part of a DNA and/or RNA molecule or primer.

Amplification. As used herein “amplification” refers to any in vitro method for increasing the number of copies of a nucleotide sequence with the use of a polymerase. Nucleic acid amplification results in the incorporation of nucleotides into a DNA and/or RNA molecule or primer thereby forming a new molecule complementary to all or a portion of a template. The formed nucleic acid molecule and its template can be used as templates to synthesize additional nucleic acid molecules. As used herein, one amplification reaction may consist of many rounds of replication. DNA amplification reactions include, for example, polymerase chain reactions (PCR). One PCR reaction may consist of 5 to 100 “cycles” of denaturation and synthesis of a DNA molecule.

Nucleotide. As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, 2′-Omethyl modified derivative, biotinylated nucleotides and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Oligonucleotide. “Oligonucleotide” refers to a synthetic or natural molecule comprising a covalently linked sequence of nucleotides which are joined by a phosphodiester, or phosphorothioate, or amido bond between the 3′ position of the deoxyribose or ribose of one nucleotide and the 5′ position of the deoxyribose or ribose of the adjacent nucleotide.

Hybridization. The terms “hybridization” and “hybridizing” refers to base pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used.

Unit. The term “unit” as used herein refers to the activity of an enzyme. When referring, for example, to a DNA polymerase, one unit of activity is the amount of enzyme that will incorporate 10 nanomoles of dNTPs into acid-insoluble material (i.e., DNA or RNA) in 30 minutes under standard primed DNA synthesis conditions.

Vector. A plasmid, phagemid, cosmid or phage DNA or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be spliced in order to bring about its replication and cloning. The cloning vector may further contain a marker suitable for use in the identification of cells transformed with the cloning vector. Markers, for example, are tetracycline resistance or ampicillin resistance.

Expression vector. A vector similar to a cloning vector but which is capable of enhancing the expression of a gene which has been cloned into it, after transformation into a host. The cloned gene is usually placed under the control of (i.e., operably linked to) certain control sequences such as promoter sequences.

Recombinant host. Any prokaryotic or eukaryotic organism or cell which contains the desired cloned genes in an expression vector, cloning vector or any DNA molecule. The term “recombinant host” is also meant to include those host cells which have been genetically engineered to contain the desired gene on the host chromosome or genome.

Host. Any prokaryotic or eukaryotic organism or cell that is the recipient of a replicable expression vector, cloning vector or any DNA molecule. The DNA molecule may contain, but is not limited to, a structural gene, a promoter and/or an origin of replication.

Promoter. A DNA sequence generally described as the 5′ region of a gene, located proximal to the start codon. At the promoter region, transcription of an adjacent gene(s) is initiated.

Gene. A DNA sequence that contains information necessary for expression of a polypeptide or protein. It includes the promoter and the structural gene as well as other sequences involved in expression of the protein.

Structural gene. A DNA sequence that is transcribed into messenger RNA that is then translated into a sequence of amino acids characteristic of a specific polypeptide.

Operably linked. As used herein means that the promoter is positioned to control the initiation of expression of the polypeptide encoded by the structural gene.

Expression. Expression is the process by which a gene produces a polypeptide. It includes transcription of the gene into messenger RNA (mRNA) and the translation of such mRNA into polypeptide(s).

Substantially Pure. As used herein “substantially pure” means that the desired purified protein or polypeptide is essentially free from contaminating cellular contaminants which are associated with the desired protein or polypeptide in nature. Contaminating cellular components may include, but are not limited to, phosphatases, exonucleases, endonucleases or undesirable DNA polymerase enzymes.

Thermostable. As used herein “thermostable” refers to a polypeptide or enzyme (e.g., DNA polymerase, nuclease, and reverse transcriptase) which is resistant to inactivation by heat. By way of example, DNA polymerases synthesize the formation of a DNA molecule complementary to a single-stranded DNA template by extending a primer in the 5′ to 3′ direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable polymerase activity is more resistant to heat inactivation than a mesophilic polymerase. However, a thermostable polymerase does not mean to refer to an enzyme which is totally resistant to heat inactivation and thus heat treatment may reduce the polymerase activity to some extent. A thermostable polymerase typically will also have a higher optimum temperature than mesophilic polymerases.

3′ to 5′ Exonuclease Activity. “3′ to 5′ exonuclease activity” is an enzymatic activity well known to the art. This activity is often associated with DNA polymerases, and is thought to be involved in a DNA replication “editing” or correction mechanism.

A “polymerase substantially reduced in 3′ to 5′ exonuclease activity” is defined herein as either (1) a mutated or modified polymerase that has about or less than 10%, or preferably about or less than 1%, of the 3′ to 5′ exonuclease activity of the corresponding unmutated, wild-type enzyme, or (2) a polymerase having a 3′ to 5′ exonuclease specific activity which is less than about 1 unit/mg protein, or preferably about or less than 0.1 units/mg protein. A unit of activity of 3′ to 5′ exonuclease is defmed as the amount of activity that solubilizes 10 nmoles of substrate ends in 60 min. at 37° C., assayed as described in the “BRL 1989 Catalogue & Reference Guide”, page 5, with HhaI fragments of lambda DNA 3′-end labeled with [³H]dTTP by terminal deoxynucleotidyl transferase (TdT). Protein is measured by the method of Bradford, Anal. Biochem. 72:248 (1976). As a means of comparison, natural, wild-type T5-DNA polymerase (DNAP) or T5-DNAP encoded by pTTQ19-T5-2 has a specific activity of about 10 units/mg protein while the DNA polymerase encoded by pTTQ19-T5-2(Exo-) (U.S. Pat. No. 5,270,179) has a specific activity of about 0.0001 units/mg protein, or 0.001% of the specific activity of the unmodified enzyme, a 105-fold reduction.

5′ to 3′ Exonuclease Activity. “5′ to 3′ exonuclease activity” is also an enzymatic activity well known in the art. This activity is often associated with DNA polymerases, such as E. coli PolI and Taq DNA polymerase.

A “polymerase substantially reduced in 5′ to 3′ exonuclease activity” is defined herein as either (1) a mutated or modified polymerase that has about or less than 10%, or preferably about or less than 1%, of the 5′ to 3′ exonuclease activity of the corresponding unmutated, wild-type enzyme, or (2) a polymerase having 5′ to 3′ exonuclease specific activity which is less than about 1 unit mg protein, or preferably about or less than 0.1 units/mg protein.

Both of the 3′ to 5′ and 5′ to 3′ exonuclease activities can be observed on sequencing gels. Active 5′ to 3′ exonuclease activity will produce nonspecific ladders in a sequencing gel by removing nucleotides from the 5′-end of the growing primers. 3′ to 5′ exonuclease activity can be measured by following the degradation of radiolabeled primers in a sequencing gel. Thus, the relative amounts of these activities, e.g., by comparing wild-type and mutant or modified polymerases, can be determined with no more than routine experimentation.

Other terms used in the fields of recombinant DNA technology and molecular and cell biology as used herein will be generally understood by one of ordinary skill in the applicable arts.

Ligands

The ligands of the present invention include a variety of compounds/molecules (including natural and synthetic) having affinity for double-stranded nucleic acids (i.e., DNA/DNA, DNA/RNA, RNA/RNA, PNA/DNA, PNA/RNA, LNA/DNA or LNA/RNA) and/or for single-stranded nucleic acids (e.g., RNA or DNA or PNA or LNA or combinations thereof) and/or single-stranded/double-stranded nucleic acid complexes, or other oligonucleotides or modified oligonucleotides (e.g., having phophorothioate linkages, 3′-Omethyl ribose bases, etc.). Thus, the ligands of the invention may be used with any natural or derivative or synthetic nucleic acid molecules in accordance with the invention. Numerous synthetic, natural and derivative nucleic acid molecules are known in the art and are routinely used as substrates in synthesis, amplification and sequencing reactions. Such nucleic acid molecules may comprise modified groups, detectable labels, derivative nucleotides, modified linkages, modified bases, modified sugars and the like. In accordance with the invention, such natural, synthetic and derivative synthesis, amplification and sequencing substrates may be used in combination with the ligands (e.g., cationic/polycationic compounds) of the invention. Such ligands may include or may be derived from any proteins, sugars, steroids, or lipids which bind to or have affinity for such nucleic acid molecules. Examples of such ligands include but are not limited to natural compounds such as histones, protamine, spermine, spermidine, and high mobility group proteins, and synthetic cationic compositions such as DEAE-dextran, polybrene, polylysine, polyhistidine, polypeptides, polyamidoamine cascade polymers or dendrimers, lipopolyamines, and polyethylenimine, and cationic lipid or liposome formulations such as “Transfectam™” (Promega), “DOTAP™” (Roche), “FUGENE 6™” (Roche), “X-treme GENE Q2™” (Roche), “GeneJammer™” (Stratagene), “GenePorter™” (Gene Therapy Systems), “Effectene™” (Quiagen), “Superfect™” (Quiagen), “LIPOFECTIN™” (Invitrogen Corporation, Life Technologies Division), “LIPOFECTACE™” (Invitrogen Corporation, Life Technologies Division), “LIPOFECTAMINE™” (Invitrogen Corporation, Life Technologies Division), “LIPOFECTAMINE 2000™” (Invitrogen Corporation, Life Technologies Division), “CELLFECTIN™” (Invitrogen Corporation, Life Technologies Division), “DMRIE-C™” (Invitrogen Corporation, Life Technologies Division), natural and synthetic peptides having a cationic charge which interact with nucleic acids such that the nucleic acid is not spliced due to the binding of the peptide, cationic detergents, and other cationic compounds described in the following patents: U.S. Pat. Nos. 4,812,449, 5,171,678, 5,186,923, 5,277,897, 5,208,036, 5,208,036 , 5,264,618, 5,279,833, 5,334,761, 4,897,355, 5,459,127, 5,650,096, 5,744,335, 5,854,224, 5,670,347, 5,869,606, 5,906,922, 5,674,908, WO 98/19709, U.S. Pat. No. 5,861,397, U.S. Pat. No. 5,670,347, WO 93/19768, WO 00/27795, WO 97/42819, EP 0846680, U.S. Pat. No. 5,830,430, WO 98/40502, WO 98/40499, WO 98/02190, and WO 99/29712.

Cationic compounds that may be used in accordance with the invention include those of Formula I:

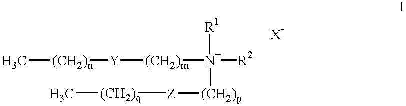

wherein R¹ and R² are independently H, C₁₋₁₀ alkyl, preferably C₁₋₆ alkyl, more preferably C₁₋₃ alkyl and Y and Z are independently members selected from the group consisting of —CH₂CH₂CH₂CH₂CH₂—, —CH═CHCH₂CH₂CH₂—, —CH₂CH═CHCH₂CH₂—, —CH₂CH₂CH═CHCH₂—, —CH₂CH₂CH₂CH═CH—, —CH═CHCH═CHCH₂—, —CH═CH₂CH₂CH═CH—, and CH₂CH═CHCH═CH—; n and q are independently integers of from 3 to 10, preferably 3 to 7; and m and p are independently integers of from 2 to 12, preferably from 4 to 9, with the proviso that the sums n+m and q+p are each integers of from 10 to 17 and X is an anion. X can be a monovalent or multivalent anion. Preferred compounds of Formula I include N,N-dioleyl-N,N-dimethylammonium chloride and N-stearyl-N-oleyl-N,N-dimethylammonium chloride. See U.S. Pat. No. 5,753,613.

Another group of cationic compounds that may be used in accordance with the invention include cationic lipids of Formula II:

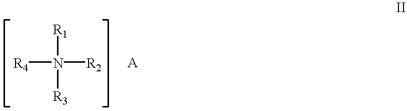

wherein

R₁ is a straight or a branched hydrocarbon chain of C₁₀₋₁₀₀ that is saturated or unsaturated;

R₂ is selected from the group consisting of a pair of electrons, hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted; R₃ and R₄, independently of one another, are selected from the group consisting of hydrogen, C₁₋₁₀₀ alkyl, preferably, C₆₋₂₂ alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted; wherein R₅ and R₆ are independently alkylene, alkenylene or alkynylene; and A is a pharmaceutically acceptable anion when R₂ is not a pair of electrons; and optionally at least one neutral lipid to form one or more lipid aggregate complexes. See U.S. Pat. No. 5,279,833.

In a preferred aspect, R₁ is a straight or a branched hydrocarbon chain of C₁₀₋₃₀ that is saturated or unsaturated. In another preferred aspect, when R₃ and R₄ in Formula II are C₁₋₃ alkyl, and one of R₁ or R₂ is an unsaturated C₁₆₋₂₀ alkyl, the other one of R₁ and R₂ is not an unsaturated or saturated C₁₆₋₂₀ alkyl. Preferably, R₁ is a straight or a branched hydrocarbon chain of C₁₀₋₃₀ that is saturated or unsaturated. Preferably, R₁ is a straight hydrocarbon chain of C₁₂₋₂₄ that is saturated or unsaturated; and R₂, R₃ and R₄ are independently selected from the group consisting of hydrogen, C₁₋₂₀ alkyl, C₂₋₂₀ alkenyl, C₂₋₂₀ alkynyl, C₄₋₂₀ heteroalkyl, C₄₋₂₀ heteroalkenyl, C₄₋₂₀ heteroalkynyl, C₆₋₁₂ aryl(C₁₋₂₀) alkyl and C₆₋₁₂ aryl, all of which can be optionally substituted. More preferably, R₁ is a straight hydrocarbon chain of C₁₄₋₂₀ that is saturated or unsaturated; R₂ is selected from the group consisting of hydrogen, C₆₋₁₈ alkyl, C₆₋₁₈ alkenyl, C₆₋₁₈ alkynyl, C₆₋₁₈ heteroalkyl, C₆₋₁₈ heteroalkenyl, C₆₋₁₈ heteroalkynyl, phenyl(C₆₋₁₈)alkyl, and phenyl; and R₃ and R₄ are independently selected from the group consisting of hydrogen, C₁₋₅ alkyl, C₂₋₆ alkenyl, C₂₋₆ alkynyl, C₂₋₅ heteroalkyl, C₂₋₅ heteroalkenyl, C₂₋₅ heteroalkynyl, phenyl(C₁₋₅)alkyl, especially benzyl, and phenyl, all of which can be optionally substituted. Another useful group of cationic lipids of Formula II include those wherein R₁ and R₂ are both C₁₀₋₂₀ saturated alkyl groups.

In another preferred aspect, the cationic lipid has the Formula III:

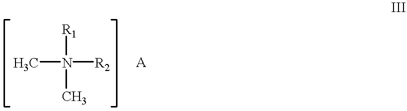

A is any compatable anion. R₁ and R₂ are defined above with respect to Formula II. These anions can be organic or inorganic. A is preferably a halogen, that is Br⁻, Cl⁻, F⁻, I⁻, or A is a sulfate, a nitrite or a nitrate. Preferred compounds include cetyldimethylethylammonium bromide and dimethyldioctadecylammonium bromide (DDAB).

In another preferred aspect, cationic compound has the Formula IV:

[chemical structure]

or an enantiomer thereof, wherein R¹ and R² are independently an alkyl, alkenyl, or alkynyl group of 6 to 24 carbon atoms, R³, R⁴ and R⁵ are independently hydrogen, alkyl of 1 to 8 carbon atoms, aryl or aralkyl of 6 to 11 carbon atoms; alternatively two or three of R³, R⁴ and R⁵ are combined with the positively charged nitrogen atom to form a cyclic structure having from 5 to 8 atoms, where, in addition to the positively charged nitrogen atom, the atoms in the structure are carbon atoms and can include one oxygen, nitrogen or sulfur atom; n is 1 to 8; and X is an anion. A preferred compound of Formula IV is N-(2,3-di(9-(Z)-octadecenyloxy))-prop-1-yl-N,N,N-trimethylammonium chloride (DOTMA). See U.S. Pat. No. 5,550,289.

Also useful to the practice of the present invention are lipids having Formula V:

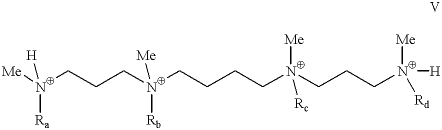

wherein the groups Rₐ, R_{b}, R_{c} and R_{d} are independently C₁₂, C₁₃, C₁₄, C₁₅, C₁₆, C₁₇, C₁₈, C₁₉, C₂₀, C₂₁ or C₂₂ straight chain alkyl or alkenyl groups. In a preferred embodiment, the longer chain lipids (C₁₈-C₂₂) are employed. Preferred compounds of Formula V include tetramethyltetrapalmitylspermine (TMTPS), tetramethyltetralaurylsperinine (TMTLS), tetramethyltetramyristylspermine (TMTMS), tetramethyltetrasterylspermine (TMTSS), and tetramethyltetraoleoylspermine (TMTOS). See WO 98/40499.

In another embodiment, the cationic lipid is a nitrogen-containing, imidazolinium-derived cationic lipid having Formula VI:

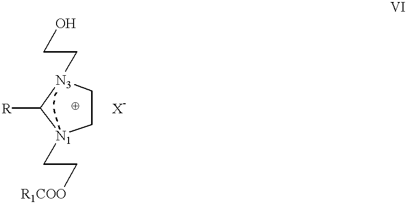

wherein each of R and R₁ independently is a straight-chain, aliphatic hydrocarbyl group of 11 to 29 carbon atoms inclusive, and X⁻ is a monovalent or multivalent anion. Optionally, R and R₁ may be substituted by a carboxyl group to give a zwitterionic compound. A preferred compound of Formnula VI is 1-(2-(oleoyloxy)ethyl)-2-oleyl-3-(2-hydroxyethyl)imidazolinium. See U.S. Pat. No. 5,830,878.

In another preferred embodiment, cationic compounds include dioctadecyl amidoglycylspennine (DOGS) and dipalmitoyl phosphatidylethanolamidospermine (DPPES). In both compounds, the anion may be trifluoroacetic acid, as described in J. Behr, et al, Proc. Natl. Acad. Sci. USA 86:6982-6986 (1989), or other anion.

In another preferred embodiment, cationic compounds that may be used in accordance with the invention include: (1-{(3-aminopropyl)-[4-(3-aminopropylamino)-butyl]-carbamoyl}-2-phenylethyl)carbamic acid 17-(1,5-dimethylhexyl)-10,13-dimethyl-2,3,4,7,8,9,10,11,12,13,14,15,16,17-tetradeca-hydro-1H-cyclopenta{a}phenanthren-3-yl ester; [1-{(3-amino-propyl)-[4-(3-amino-propylamino)butyl]carbamoyl}-2(4-hydroxyphenyl)-ethyl]carbamic acid 17-(1,5-dimethylhexyl)-10,13-dimethyl-2,3,4,7,8,9,10,11,12,13,14,15,-16,17-tetradecaahydro-1H-cyclopenta{a}-phenanthren-3-yl ester; {5-amino-5-[(4-aminobutyl)-(3-amino-propyl)carbamoyl]pentyl}carbamic acid 17-(1,5-dimethylhexyl)-10,13 -dimethyl-2,3,4,7,8,9,10,11,12,13,14,15,16,17-tetra-decahydro-1H-cyclopenta[α]phenanthren-3-yl ester); (5-amino-5{(3-amino-propyl)-[4-(3-aminopropyl-aminobutyl]carbamoyl}-pentyl)carbamic acid 17-(1,5-dimethylhexyl)-10,13-dimethyl-2,3,4,7,8,9,10,11,12,13,14,15,16,17-tetradecahydro-1H-cyclopenta[α]phenanthren-3-yl ester; and (5-amino-1-{3-aminopropyl)-[4-(3-aminopropylamino)butyl]carbamoyl}pentyl)-carbamic acid 17-(1,5-dimethyl-hexyl)-10,13-dimethyl-2,3,4,7,8,9,10,11,12,13,14,15,-16,17-tetradecahydro-1H-cyclopenta[α]phenanthren-3-yl ester. See U.S. Pat. No. 5,948,925.

In another preferred embodiment, cationic compounds that may be used in accordance with the invention include: cholesteryl-3β-carboxyl-amidoethylenetrimethylammonium iodide, 1-dimethylamino-3-trimethyl-ammonio-DL-2-propyl-cholesteryl carboxylate iodide, cholesteryl-3β-carboxyamidoethyleneamine, cholesteryl-3β-oxysuccinamidoethylenetri-methylammonium iodide, 1-dimethylamino-3-trimethylammonio-DL-2-propyl-cholesteryl-3β-oxysuccinate iodide, 2-[(2-trimethylammonio)-ethylmethylamino]ethyl-cholesteryl-3β-oxysuccinate iodide, 3β[N-(N′,N′-dimethylaminoethane)carbamoyl]cholesterol, and 3β-[N-(polyethyleneimine)-carbamoyl]cholesterol. See U.S. Pat. No. 5,283,185.

In another preferred embodiment, cationic compounds that may be used in accordance with the invention include: spermine cholesterol carbamate, N⁴-spermine cholesteryl carbamate, N,N-dioctadecyllysineamide, lysine 3-N-dihydrocholesteryl carbamate, and N¹,N¹-dioctadecyl-1,2,6-triaminohexane. See U.S. Pat. No. 5,650,096.

In another preferred embodiment, the cationic compound is a polyamine having Formula VII:

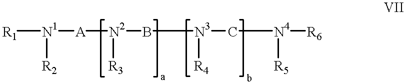

or its possible stereoisomers or a salt thereof with a pharmaceutically acceptable acid wherein:

R₁ and R₄ may be the same or different and are alkyl, aryl, aryl alkyl or cycloalkyl, optionally having an alkyl chain interrupted by at least one etheric oxygen atom;

R₂ and R₃ may be the same or different and are R₁, R₄ or H;

N₁, N₂, N₃ and N₄ are nitrogen atoms capable of protonation at physiological pHs;

A, B, and C may be the same or different and are bridging groups which effectively maintain the distance between the nitrogen atoms such that the polyamine:

(i) is capable of uptake by a target cell upon administration of the polyamine to a human or non-human animal or is capable of binding to at least one polyamine site of a receptor located within or on the surface of a cell upon administration of the polyamine to a human or non-human animal; and

(ii) upon uptake by the target cell, competitively binds via an electrostatic interaction between the positively charged nitrogen atoms to biological counter-anions;

the polyamine, upon binding to the biological counter-anion in the cell, functions in a manner biologically different than the intracellular polyamines, and further wherein at least one of said bridging groups A, B and C contains at least one —CH(OH)— group which is not alpha- to either of the nitrogen atoms. Preferred compounds of Formula VII include diethylnorspermine (DENSPM), MENSPM, DENSPM, DIPNSPM, DMSPM, MESPM, DESPM, DPSPM, FDESPM, DMHSPM, MEHSPM, DEHSPM, DIPHSPM, ETBHSPM, DTBHSPM, DE(3,4,4), DE(4,5,4), PIP(3,4,3) PYR(3,3,3), PIP(4,4,4), PYR(4,4,4), PIP(5,4,5), BAHSPM, CHX(4,4,4)-trans, and CHX(3,4,3)-trans. See U.S. Pat. No. 5,962,533.

The invention further contemplates the use of a cationic lipid compound of the Formula VIII:

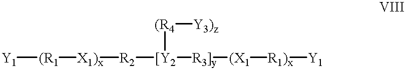

wherein:

each of x, y and z are independently an integer from 0 to about 100;

each X₁ is independently —O—, —S—, —NR₅—, —C(═X₂) —, —C(═X₂)—N(R₅)—, —N(R₅)—C(═X₂)—, —C(═X₂)—O—, —O—C(═X₂)— or —X₂—(R₅X₂)P(═X₂)—X₂—;

each X₂ is independently O or S;

each Y₁ is independently a phosphate residue, N(R₆)ₐ—, S(R₆)ₐ—, P(R₆)ₐ— or —CO₂R₆, wherein a is an integer from 1 to 3;

each Y₂ is independently —N(R₆)_{b}—, —S(R₆)_{b}— or P(R₆)_{b}—, wherein b is an integer from 0 to 2;

each Y₃ is independently a phosphate residue, N(R₆)ₐ—, S(R₆)ₐ—, P(R₆)ₐ— or —CO₂R₆, wherein a is an integer from 1 to 3;

each of R₁, R₂, R₃ and R₄ is independently alkylene of 2 to about 20 carbons;

each R₅ is independently hydrogen or alkyl of 1 to about 10 carbons; and

each R₆ is independently —[R₇—X₃]_{c}—R₈ or —R₉—[X₄—R₁₀]_{d}—Q, wherein:

each of c and d is independently an integer from 0 to about 100;

each Q is independently a phosphate residue, —N(R₁₁)_{q}—, S(R₁₁)_{q}—, P(R₁₁)_{q}— or —CO₂R₁₁, wherein q is an integer from 1 to 3;

each of X₃ and X₄ is independently —O—, —S—, —NR₅—, —C(═X₂)—, —C(═X₂)—N(R₅)—, —N(R₅)—C(═X₂)—, —C(═X₂)—O—, —O—C(═X₂)— or —X₂—(R₅X₂)P(═X₂)—X₂—;

each R₇ is independently alkylene of 2 to about 20 carbons;

each R₈ is independently hydrogen or alkyl of 1 to about 60 carbons;

each of R₉ and R₁₀ is independently alkylene of 2 to about 20 carbons; and

each R₁₁ is independently —[R₇—X₃]_{c}—R₈ or —R₉—[X₄—R₁₀]_{d}—W, wherein:

each W is independently a phosphate residue, —N(R₁₂)_{w}—, S(R₁₂)_{w}—, P(R₁₂)_{w}— or —CO₂R₁₂, wherein w is an integer from 1 to 3; and

R₁₂ is —[R₇—X₃]_{c}—R₈, with the proviso that the compound of formula (I) comprises at least two quaternary salts. Preferred compounds of Formula VIII include N,N′-bis(dodecylaminocarbonylmethylene)-N,N′-bis(β-N,N,N-trimethylammoniumethylaminocarbonylmethylene)-N,N′-dimethyl-ethylenediamine tetraiodide (EDTA-LA-TMA tetraiodide); N,N′-bis(dodecylaminocarbonylmethylene)ethylenediamine-N,N′-diacetic acid (EDTA-LA); N,N″-bis(hexadecylaminocarbonylmethylene)-N,N′,N″-tris(β-N,N,N-trimethylammoniumethylaminocarbonylmethylene)-N,N′,N″-tri-methyldiethylenetriamine hexaiodide (DTPA-HA-TME hexaiodide); N,N′-bis(dodecylaminocarbonylmethylene)-N,N′-bis(β-N,N,N-trimethylammoniumethylaminocarbonylmethylene)-N,N′-dimethyl cyclohexylene-1,4-diamine tetraiodide (CDTA-LA-TMA tetraiodide); 1,1,7,7-tetra(β-N,N,N,N-tetra-methylammoniumethylaminocarbonylmethylene)-4-hexadecylaminocarbonylmethylene-N,N′,N″-trimethyl-1,4,7-triazaheptane heptaiodide (DTPA-MHA-TTMA heptaiodide); N,N′-bis(dodecyloxycarbonylmethylene)-N,N′-bis(β-N,N,N-trimethylammoniumethylaminocarbonylmethylene)ethylenediamine diiodide; N,N,N″,N″-tetra(β-N,N,N-trimethylammoniumethylaminocarbonylmethylene)-N′-(1,2-dioleoylglycero-3-phosphoethanolaminocarbonylmethylene)diethylenetriamine tetraiodide; N,N′-bis(hexadecylaminocarbonylmethylene-N,N′-bis(trimethylammoniumethylaminocarbonylmethylene)-ethylenediamine diiodide; N,N′-bis(hexadecyloxycarbonylmethylene)-N-(β-N,N,N-trimethylammoniumethylaminocarbonylmethylene)-N-methyl-N′-(carboxymethylene)ethylenediamine diiodide; and N,N′-bis(hexadecylaminocarbonylmethylene)-N,N′-bis(β-N,N,N-trimethylammoniumethylaminocarbonylmethylene)-N,N-dimethylethylenediamine tetraiodide). See U.S. Pat. No. 5,830,430.

The invention also contemplates the use of cationic compounds of Formula IX:

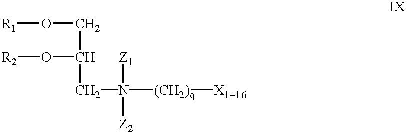

wherein R₁ and R₂ separately or together are C₁₋₂₃ alkyl or

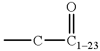

alkyl or alkenyl, q is 1 to 6,

Z₁ and Z₂ separately or together are H or unbranched alkyl C₁₋₆,

X₁ is —(CH₂)ₙBr, Cl, F or I n=0-6 or

X₂ is —(CH₂)ₙNH₂ n=0-6 or

X₃ is —NH—(CH₂)ₘNH₂ m=2-6 or

X₄ is —NH—(CH₂)₃NH—(CH₂)₄—NH₂ or

X₅ is —NH—CH₂)₃—NH(CH₂)₄—NH(CH₂)₃—NH₂.

X₆ is

X₇ is

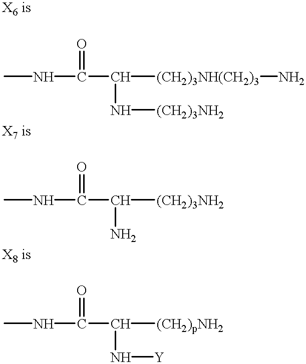

X₈ is

where p is 2-5, Y is H or other groups attached by amide or alkyl amino group or

X₉ is a polyamine, e.g., polylysine, polyarginine, polybrene, histone or protamine or

X₁₀ is a reporter molecule, e.g.,

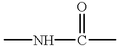

fluorescein, biotin, folic acid or PPD, or

X₁₁ is a polysaccharide or substituted polysaccharide, or

X₁₂ is a protein or

X₁₃ is an antibody or

X₁₄ is an amine or halide reactive group or

X₁₅ is —(CH₂)ᵣ—SH where r is 0-6 or

X₁₆ is —(CH₂)ₛ—S—S—(CH₂)ₜ—NH₂ where s is 0-6 and t is 2-6. See WO 94/27435.

The complexes may further comprise at least one neutral lipid. Examples of neutral lipids which can be used include, for example, diacylphosphatidylcholine, diacylphosphatidylethanolamine, ceramide, sphingomyelin, phosphatidic acid, and cholesterol. Preferably, the neutral lipid is selected from the group consisting of diacylphosphatidylcholine, such as dioleyphosphatidylcholine, dipalmitoylphosphatidylcholine, palmitoyloleylphosphatidylcholine, lecithin and lysolecithin, diacylphosphatidylethanolamine, ceramide, sphingomyelin, and cholesterol. More preferably, the neutral lipid is a diacylphosphatidylethanolamine having 10-24 carbon atoms in the acyl group. More preferably the acyl groups are lauroyl, myristoyl, heptadecanoyl, palmitoyl, stearoyl or oleyl. Especially, the neutral lipid is dioleylphosphatidylethanolamine (DOPE), palmitoyloleylphosphatidylethanolamine, diheptadecanoylphosphatidylethanolamine, dilauroylphosphatidylethanolamine, dimyristoylphosphatidylethanolamine, distearoylphosphatidylethanolamine, beta-linoleyl-gamma-palmitoylphosphatidylethanolamine, and beta-oleyl-gamma-palmitoylphosphatidylethanolamine, specifically dioleylphosphatidyl-ethanolamine (DOPE).

The ratio of the cationic lipid to a neutral lipid can be widely varied depending on the particular cationic lipid employed. For example, the ratio can be from about 1:10 to about 10:1, preferably from about 1:7 to about 7:1, more preferably from about 1:5 to about 5:1, more preferably from about 2.5:1 to about 1:2.5.

Useful alkyl groups include straight-chained and branched C₁₋₁₈ alkyl groups, preferably C₁₋₁₀ alkyl groups, more preferably C₁₋₅ alkyl groups. Typical C₁₋₁₈ alkyl groups include methyl, ethyl, propyl, isopropyl, butyl, sec-butyl, tert-butyl, 3-pentyl, hexyl, octyl, decyl, dodecyl, tetradecyl, hexadecyl and octadecyl groups.

Useful alkenyl groups are C₂₋₁₈ alkenyl groups, preferably C₂₋₁₀ alkenyl, more preferably C₂₋₆ alkenyl groups. Typical C₂₋₁₈ alkenyl groups include ethenyl, propenyl, isopropenyl, butenyl, sec-butenyl, hexenyl, octeneyl, decenyl, dodecenyl, tetradecenyl, especially 9-tetradecenyl, hexadecenyl, especially 9-hexadecenyl, and octadecenyl, especially 9-octadecenyl, groups.

Useful alkynyl groups are C₂₋₁₈ alkynyl groups, preferably C₂₋₁₀ alkynyl, more preferably C₂₋₆ alkynyl groups. Typical C₂₋₁₈ alkynyl groups include ethynyl, propynyl, butynyl, 2-butynyl, hexynyl, octynyl, decynyl, dodecynyl, tetradecynyl, hexadecynyl, and octadecynyl groups.

Typical heteroalkyl groups include any of the above-mentioned C₁₋₁₈ alkyl groups having one or more CH₂ groups replaced with O or S.

Typical heteroalkenyl groups include any of the above-mentioned C₂₋₁₈ alkenyl groups having one or more CH₂ groups replaced with O or S.

Typical heteroalkynyl groups include any of the above-mentioned C₂₋₁₈ alkynyl groups having one or more CH₂ groups replaced with O or S.

Typically alkylaminoalkyl groups are R₇—NH—R₈, wherein R₇ and R₈ are alkylene groups as defined above.

Useful aryl groups are C₆₋₁₄ aryl, especially C₆₋₁₀ aryl. Typical C₆₋₁₄ aryl groups include phenyl, naphthyl, phenanthryl, anthracyl, indenyl, azulenyl, biphenyl, biphenylenyl and fluorenyl groups.

Useful arylalkyl groups include any of the above-mentioned C₁₋₁₈ alkyl groups substituted by any of the above-mentioned C₆₋₁₄ aryl groups. Useful values include benzyl, phenethyl and naphthylmethyl.

Useful arylalkenyl groups include any of the above-mentioned C₂₋₁₈ alkenyl groups substituted by any of the above-mentioned C₆₋₁₄ aryl groups.

Useful arylalkynyl groups include any of the above-mentioned C₂₋₁₈ alkynyl groups substituted by any of the above-mentioned C₆₋₁₄ aryl groups. Useful values include phenylethynyl and phenylpropynyl.

Useful halo or halogen groups include fluorine, chlorine, bromine and iodine.

Useful haloalkyl groups include C₁₋₁₀ alkyl groups substituted by one or more fluorine, chlorine, bromine or iodine atoms, e.g. fluoromethyl, difluoromethyl, trifluoromethyl, pentafluoroethyl, 1,1-difluoroethyl and trichloromethyl groups.

Useful hydroxyalkyl groups include C₁₋₁₀ alkyl groups substituted by hydroxy, e.g. hydroxymethyl, hydroxyethyl, hydroxypropyl and hydroxybutyl groups.

Useful alkoxy groups include oxygen substituted by one of the C₁₋₁₀ alkyl groups mentioned above.

Useful alkylthio groups include sulfur substituted by one of the C₁₋₁₀ alkyl groups mentioned above.

Useful acylamino groups are any acyl group, particularly C₂₋₆ alkanoyl or C₆₋₁₀ aryl(C₂₋₆)alkanoyl attached to an amino nitrogen, e.g. acetamido, propionamido, butanoylamido, pentanoylamido, hexanoylamido, and benzoyl.

Useful acyloxy groups are any C₁₋₆ acyl (alkanoyl) attached to an oxy (—O—) group, e.g. acetoxy, propionoyloxy, butanoyloxy, pentanoyloxy, hexanoyloxy and the like.

Useful alkylamino and dialkylamino groups are —NHR₉ and —NR₉R₁₀, wherein R₉ and R₁₀ are C₁₋₁₀ alkyl groups.

Aminocarbonyl group is —C(O)NH₂.

Useful alkylthiol groups include any of the above-mentioned mentioned C₁₋₁₀ alkyl groups substituted by a —SH group.

A carboxy group is —COOH.

An ureido group is —NH—C(O)—NH₂.

An amino group is —NH₂.

Optional substituents on the R groups include any one of halogen, halo(C₁₋₆) alkyl, C₁₋₆ alkyl, C₂₋₆ alkenyl, C₂₋₆ alkynyl, hydroxy(C₁₋₆)alkyl, amino(C₁₋₆)alkyl, carboxy(C₁₋₆)alkyl, alkoxy(C₁₋₆)alkyl, nitro, amino, ureido, acylamino, hydroxy, thiol, acyloxy, alkoxy, carboxy, aminocarbonyl, and C₁₋₆ alkylthiol groups mentioned above. Preferred optional substituents include: hydroxy(C₁₋₆)alkyl, amino(C₁₋₆)alkyl, hydroxy, carboxy, nitro, C₁₋₆ alkyl, alkoxy, thiol and amino.

As will be recognized, other ligands (natural, unnatural, modified etc.) may be selected and used in accordance with the invention. Such selection may be accomplished by double-stranded and/or single-stranded and/or single-stranded/double-stranded nucleic acid complex nucleic acid binding studies and/or nucleic acid synthesis inhibition assays. Preferred ligands are those which are polycationic and preferably form complexes with nucleic acids sufficiently stable to inhibit unwanted enzymatic activity under certain conditions. Preferably, the complexes prevent polymerase and/or nuclease activity. In one aspect, transfection agents which complex with nucleic acids and allow transfection in a cell may be used in accordance with the invention. Cationic or polycationic compounds/molecules/compositions for use in the invention may be synthesized by well known techniques or obtained commercially.

Ligands (e.g., cationic compounds/molecules/compositions) of the present invention are preferably used at a final concentration in a synthesis, sequencing or amplification reaction sufficient to prevent or inhibit such synthesis, sequencing or amplification in the presence of a polymerase or reverse transcriptase enzyme. The ratio of ligands of the invention to polymerase or reverse transcriptase may vary depending on the polymerase or reverse transcriptase and ligand used. The molar ratio of ligands (e.g., cationic compounds/molecules/compositions) to polymerase/reverse transcriptase enzyme for a synthesis, sequencing or amplification reaction may range from about 0.001-1,000,000:1; about 0.01-100,000:1; about 0.1-10,000:1; about 1-1,000:1; about 1-50:1; about 1-10:1; about 1-5:1; or about 1-2:1. Of course, other suitable ratios of such ligand to polymerase/reverse transcriptase suitable for use in the invention will be apparent to one or ordinary skill in the art or determined with no more than routine experimentation.

Methods of Nucleic Acid Synthesis

The ligands (particularly cationic compounds/molecules/compositions) of the invention may be used in methods for the synthesis of nucleic acids. In particular, it has been discovered that the present ligands reduce nonspecific nucleic acid synthesis, particularly in amplification reactions such as the polymerase chain reaction (PCR). The present cationic compounds/molecules/compositions may therefore be used in any method requiring the synthesis of nucleic acid molecules, such as DNA (including cDNA) and RNA molecules. Methods in which the ligands (e.g., cationic compounds/molecules/compositions) of the invention may advantageously be used include, but are not limited to, nucleic acid synthesis methods, nucleic acid amplification methods, including “hot-start” synthesis or amplification where the reaction is set up at a temperature below which the ligands dissociate, or is denatured or inactivated and then the reaction is initiated by elevating the temperature (or changing other reaction conditions) to dissociate the ligands (e.g., cationic compounds/molecules/compositions) from the nucleic acid or denature or inactivate the ligand, thus allowing nucleic acid synthesis or amplification to take place.

Nucleic acid synthesis methods according to this aspect of the invention may comprise one or more steps. For example, the invention provides a method for synthesizing one or more nucleic acid molecules comprising (a) mixing one or more nucleic acid templates with one or more primers and the above-described ligands (e.g., polycationic or cationic compounds/molecules/compositions) of the present invention and one or more enzymes having polymerase or reverse transcriptase activity to form a mixture; (b) incubating the mixture under conditions sufficient to inhibit nucleic acid synthesis; and (c) incubating the mixture under conditions sufficient to make one or more first nucleic acid molecules complementary to all or a portion of the templates. According to this aspect of the invention, the nucleic acid templates may be DNA molecules such as a cDNA molecule or library, or RNA molecules such as a mRNA molecule (or a population of mRNA molecules), or any other derivative thereof Conditions sufficient to allow synthesis such as pH, temperature, ionic strength, and incubation times may be optimized by those skilled in the art.

Furthermore, the enzymes having polymerase activity for use in the invention (e.g., DNA polymerases, RNA polymerases and reverse transcriptases) may be obtained commercially, for example from Invitrogen Corporation, Life Technologies Division (Rockville, Md.), Perkin-Elmer (Branchburg, N.J.), New England BioLabs (Beverly, Mass.) or Boehringer Mannheim Biochemicals (Indianapolis, Ind.). Enzymes having reverse transcriptase activity for use in the invention may be obtained commercially, for example from Invitrogen Corporation, Life Technologies Division (Rockville, Md.), Pharmacia (Piscataway, N.J.), Sigma (Saint Louis, Mo.) or Boehringer Mannheim Biochemicals (Indianapolis, Ind.). Alternatively, polymerases or reverse transcriptases may be isolated from their natural viral or bacterial sources according to standard procedures for isolating and purifying natural proteins that are well-known to one of ordinary skill in the art (see, e.g., Houts, G. E., et al., J. Virol. 29:517 (1979)). In addition, such polymerases/reverse transcriptases may be prepared by recombinant DNA techniques that are familiar to one of ordinary skill in the art (see, e.g., Kotewicz, M. L., et al., Nucl. Acids Res. 16:265 (1988); Soltis, D. A., and Skalka, A. M., Proc. Natl. Acad. Sci. USA 85:3372-3376 (1988)). Examples of enzymes having polymerase activity and reverse transcriptase activity may include any DNA polymerases including, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™ DNA polymerase, Pyrococcus furiosus (Pfa) DNA polymerase, DEEPVENT™ DNA polymerase, Pyrococcus woosii (Pwo) DNA polymerase, Pyrococcus sp KOD2 (KOD) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™ DNA polymerase, Methanobacterium thermoautotrophicum (Mth) DNA polymerase, mycobacterium DNA polymerase (Mtb, Mlep), E. coli pol I DNA polymerase, T5 DNA polymerase, T7 DNA polymerase, and generally pol I, pol III, Family A, Family B and Family C type DNA polymerase and mutants, variants and derivatives thereof. RNA polymerases such as T3, T5 and SP6 and mutants, variants and derivatives thereof may also be used in accordance with the invention. Mutations which increase DNA affinity have been described Polesky et al., 1990, J. Biol. Chem. 265, 14579-14591. It would be within the skill of a person in the art to alter the polypeptides described above for a desired purpose.

The nucleic acid polymerases used in the present invention may be mesophilic or thermophilic, and are preferably thermophilic. Preferred mesophilic DNA polymerases include Pol I family of DNA polymerases (and their respective Klenow fragments) any of which may be isolated from organisms such as E. coli, H. influenzae, D. radiodurans, H. pylori, C. aurantiacus, R. prowazekii, T. pallidum, Synechocystis sp., B. subtilis, L. lactis, S. pneumoniae, M. tuberculosis, M. leprae, M. smegmatis, Bacteriophage L5, phi-C31 , T7, T3, T5, SP01, SP02, mitochondrial from S. cerevisiae MIP-1, and eukaryotic C. elegans, and D. melanogaster (Astatke, M. et al., 1998, J. Mol. Biol. 278, 147-165), and Family A, Family B, Family C and pol III type DNA polymerase isolated for any sources, and mutants, derivatives or variants thereof, and the like. Preferred thermostable DNA polymerases that may be used in the methods and compositions of the invention include Taq, Tne, Tma, Pfu, Tfl, Tth, Stoffel fragment, VENT™ and DEEPVENT™ DNA polymerases, and mutants, variants and derivatives thereof which have preferably been modified such that they have reduced, substantially reduced or no exonuclease activity (U.S. Pat. No. 5,436,149; U.S. Pat. 4,889,818; U.S. Pat. Nos. 4,965,188; 5,079,352; 5,614,365; 5,374,553; 5,270,179; 5,047,342; 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nucl. Acids Res. 22(15):3259-3260 (1994)).

Reverse transcriptases for use in this invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al, Science 239:487-491 (1988); U.S. Pat. Nos. 4,889,818 and 4,965,188), Tne DNA polymerase (WO 96/10640 and WO 97/09451), Tma DNA polymerase (U.S. Pat. No. 5,374,553) and mutants, variants or derivatives thereof (see, e.g., WO 97/09451 and WO 98/47912). Preferred enzymes for use in the invention include those that have reduced, substantially reduced or eliminated RNase H activity. By an enzyme “substantially reduced in RNase H activity” is meant that the enzyme has less than about 20%, more preferably less than about 15%, 10% or 5%, and most preferably less than about 2%, of the RNase H activity of the corresponding wildtype or RNase H+ enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al, Nucl. Acids Res. 16:265 (1988) and in Gerard, G. F., et al., FOCUS 14(5):91 (1992), the disclosures of all of which are fully incorporated herein by reference. Particularly preferred polypeptides for use in the invention include, but are not limited to, M-MLV H⁻ reverse transcriptase, RSV H⁻ reverse transcriptase, AMV H⁻ reverse transcriptase, RAV (rous-associated virus) H⁻ reverse transcriptase, MAV (myeloblastosis-associated virus) H⁻ reverse transcriptase and HIV H⁻ reverse transcriptase. (See U.S. Pat. No. 5,244,797 and WO 98/47912). It will be understood by one of ordinary skill, however, that any enzyme capable of producing a DNA molecule from a ribonucleic acid molecule (i.e., having reverse transcriptase activity) may be equivalently used in the compositions, methods and kits of the invention.

In accordance with the invention, the input or template nucleic acid molecules or libraries may be prepared from populations of nucleic acid molecules obtained from natural sources, such as a variety of cells, tissues, organs or organisms. Cells that may be used as sources of nucleic acid molecules may be prokaryotic (bacterial cells, including those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces) or eukaryotic (including fungi (especially yeast's), plants, protozoans and other parasites, and animals including insects (particularly Drosophila spp. cells), nematodes (particularly Caenorhabditis elegans cells), and mammals (particularly human cells)).

Once the starting cells, tissues, organs or other samples are obtained, nucleic acid molecules (such as DNA, RNA (e.g., mRNA or poly A+ RNA) molecules) may be isolated, or cDNA molecules or libraries prepared therefrom, by methods that are well-known in the art (See, e.g., Maniatis, T., et al., Cell 15:687-701 (1978); Okayama, H., and Berg, P., Mol. Cell. Biol. 2:161-170 (1982); Gubler, U., and Hoffman, B. J., Gene 25:263-269 (1983)).

In the practice of a preferred aspect of the invention, a first nucleic acid molecule may be synthesized by mixing a nucleic acid template obtained as described above, which is preferably a DNA molecule or an RNA molecule such as an mRNA molecule or a polyA+ RNA molecule, with one or more of ill the above-described ligands of the invention (or various combinations thereof) to form a mixture. Synthesis of a first nucleic acid molecule complementary to all or a portion of the nucleic acid template is preferably accomplished after raising the temperature of the reaction and denaturing or inactivating or dissociating the ligand (e.g., cationic compounds/molecules/compositions) of the present invention thereby freeing the nucleic acid synthesis substrate (e.g., double-stranded primer/template hybrid, and single-stranded primers and templates) and favoring the reverse transcription (in the case of an RNA template) and/or polymerization of the input or template nucleic acid molecules. Such synthesis is preferably accomplished in the presence of nucleotides (e.g., deoxyribonucleoside triphosphates (dNTPs), dideoxyribonucleoside triphosphates (ddNTPs) or derivatives thereof).

Of course, other techniques of nucleic acid synthesis in which the ligand (e.g., cationic compounds/molecules/compositions) may be advantageously used will be readily apparent to one of ordinary skill in the art.

Amplification and Sequencing Methods

In other aspects of the invention, the ligand (e.g., cationic compounds/molecules/compositions) of the invention may be used in methods for amplifying or sequencing nucleic acid molecules. Nucleic acid amplification methods according to this aspect of the invention may additionally comprise the use of one or more polypeptides having reverse transcriptase activity, in methods generally known in the art as one-step (e.g., one-step RT-PCR) or two-step (e.g., two-step RT-PCR) reverse transcriptase-amplification reactions. For amplification of long nucleic acid molecules (i.e., greater than about 3-5 Kb in length), a combination of DNA polymerases may be used, as described in WO 98/06736 and WO 95/16028.

Amplification methods according to this aspect of the invention may comprise one or more steps. For example, the invention provides a method for amplifying a nucleic acid molecule comprising (a) mixing a nucleic acid template with one or more of the ligand (e.g., cationic compounds/molecules/compositions) of the invention (or various combinations of the ligands described herein) to form a mixture; and (b) incubating the mixture under conditions sufficient to allow the enzyme with polymerase activity to amplify a nucleic acid molecule complementary to all or a portion of the template. In a preferred aspect, the conditions favoring synthesis dissociates the ligand (e.g., cationic compounds/molecules/compositions) from the nucleic acid or denatures or inactivates the ligand (e.g., cationic compounds/molecules/compositions) of the invention. The invention also provides nucleic acid molecules amplified by such methods.

General methods for amplification and analysis of nucleic acid molecules or fragments are well-known to one of ordinary skill in the art (see, e.g., U.S. Pat. Nos. 4,683,195; 4,683,202; and 4,800,159; Innis, M. A., et al., eds., PCR Protocols: A Guide to Methods and Applications, San Diego, Calif.: Academic Press, Inc. (1990); Griffin, H. G., and Griffin, A. M., eds., PCR Technology: Current Innovations, Boca Raton, Fla.: CRC Press (1994)). For example, amplification methods which may be used in accordance with the present invention include PCR (U.S. Pat. No. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822).

Typically, these amplification methods comprise: (a) contacting the nucleic acid sample with one or more ligand (e.g., cationic compounds/molecules/compositions) of the present invention, one or more polypeptides having nucleic acid polymerase activity in the presence of one or more primer sequences, and (b) amplifying the nucleic acid sample to generate a collection of amplified nucleic acid fragments, preferably by PCR or equivalent automated amplification technique, and (c) optionally separating the amplified nucleic acid fragments by size, preferably by gel electrophoresis, and analyzing the gels for the presence of nucleic acid fragments, for example by staining the gel with a nucleic acid-binding dye such as ethidium bromide.

Following amplification or synthesis by the methods of the present invention, the amplified or synthesized nucleic acid fragments may be isolated for further use or characterization. This step is usually accomplished by separation of the amplified or synthesized nucleic acid fragments by size and/or by any physical or biochemical means including gel electrophoresis, capillary electrophoresis, chromatography (including sizing, affinity and immunochromatography), density gradient centrifugation and immunoadsorption. Separation of nucleic acid fragments by gel electrophoresis is particularly preferred, as it provides a rapid and highly reproducible means of sensitive separation of a multitude of nucleic acid fragments, and permits direct, simultaneous comparison of the fragments in several samples of nucleic acids. One can extend this approach, in another preferred embodiment, to isolate and characterize these fragments or any nucleic acid fragment amplified or synthesized by the methods of the invention. Thus, the invention is also directed to isolated nucleic acid molecules produced by the amplification or synthesis methods of the invention.

In this embodiment, one or more of the amplified or synthesized nucleic acid fragments are removed from the gel which was used for identification (see above), according to standard techniques such as electroelution or physical excision. The isolated unique nucleic acid fragments may then be inserted into standard vectors, including expression vectors, suitable for transfection or transformation of a variety of prokaryotic (bacterial) or eukaryotic (yeast, plant or animal including human and other mammalian) cells. Alternatively, nucleic acid molecules produced by the methods of the invention may be further characterized, for example by sequencing (i.e., determining the nucleotide sequence of the nucleic acid fragments), by methods described below and others that are standard in the art (see, e.g., U.S. Pat. No. Nos. 4,962,022 and 5,498,523, which are directed to methods of DNA sequencing).

Nucleic acid sequencing methods according to the invention may comprise one or more steps. For example, the invention provides a method for sequencing a nucleic acid molecule comprising (a) mixing a nucleic acid molecule to be sequenced with one or more primers, one or more of the above-described ligand (e.g., cationic compounds/molecules/compositions) of the invention (or various combinations thereof), one or more nucleotides, one or more terminating agents (such as a dideoxynucleotide), and one or more enzymes with polymerase activity and/or exonuclease activity to form a mixture; (b) incubating the mixture under conditions sufficient to synthesize a population of molecules complementary to all or a portion of the molecule to be sequenced; and (c) separating the population to determine the nucleotide sequence of all or a portion of the molecule to be sequenced.

Nucleic acid sequencing techniques which may employ in the present invention include dideoxy sequencing methods such as those disclosed in U.S. Pat. Nos. 4,962,022 and 5,498,523.

Transformation/Transfection of Hosts or Host Cells

The present invention also provides methods for introducing nucleic acid molecules into one or more hosts or host cells. Since the ligand (e.g., cationic or polycationic compounds/molecules/compositions) of the invention may serve as transfection/transformation agents or DNA condensing agents, the invention also facilitates the introduction of nucleic acid molecules into one or more host cells. Accordingly, nucleic acid molecules synthesized or amplified in accordance with the invention in the presence of ligand (e.g., cationic compounds/molecules/compositions) can be used directly for introduction into host cells without the need to separately add transfection/transfection agents, although other agents can be added in accordance with the invention to facilitate the introduction of nucleic acid molecules. Thus, the invention relates to a method for introducing one or more nucleic acid molecules in a host or host cells comprising: (a) synthesizing or amplifying one or more nucleic acid molecules in the presence of one or more ligand (e.g., cationic or polycationic compounds/molecules/compositions) of the invention (or various combinations of the ligands described herein); and (b) introducing said synthesized or amplified nucleic acid molecules in one or more host or host cells in the presence of at least one of said ligands.

Introduction of nucleic acid molecules into host or host cells may be accomplished by standard procedures and techniques well known in the art. Depending on the type of host or cell and the type of ligand (e.g., cationic or polycationic compounds/molecules/compositions) used, different procedures may be used which will be recognized by one or ordinary skill in the art. In accordance with the invention, prokaryotic (such as gram negative and gram positive bacteria including E. coli, B subtilis, S. pneumoniae etc.) or eukaryotic (yeast, plant or animal including human or other mammalian) hosts or host cells can be transfected or transformed with nucleic acid molecules in accordance with the invention. A variety of well techniques including electroporation, transformation of chemically competent cells, transfection and like may be used in accordance with the invention.

Kits

The present invention also provides kits for use in the synthesis, amplification, or sequencing of a nucleic acid molecule. Kits according to this aspect of the invention may comprise one or more containers, such as vials, tubes, ampules, bottles and the like, which may comprise one or more of the ligands (particularly cationic compounds/molecules/compositions) of the invention.

The kits of the invention may comprise one or more of the following components: (i) one or more ligands (particularly cationic compositions of the invention), (ii) one or more polymerases and/or reverse transcriptases, (iii) one or more suitable buffers, (iv) one or more nucleotides, (v) one or more primers; (vi) one or more templates, and (vii) one or more hosts or host cells (which may be cells competent for introduction of nucleic acid molecules), and (viii) instructions for carrying out the methods of the invention.

Compositions

The present invention also relates to compositions prepared for carrying out the synthesis, amplification or sequencing methods of the invention, for carrying out the nuclease protection methods of the invention and for introducing nucleic acid molecules into hosts or host cells according to the invention. Additionally, the invention relates to compositions made during or after carrying out such methods of the invention. In a preferred aspect, a composition of the invention comprise one or more of the ligands (particularly cationic compounds/molecules/compositions) of the invention. Such compositions may further comprise one or more components selected from the group consisting of: (i) one or more polymerases and/or reverse transcriptases, (ii) one or more suitable buffers, (iii) one or more nucleotides, (iv) one or more templates, (v) one or more primers, (vi) one or more templates/primer complexes, (vii) one or more nucleic acid molecules made by the synthesis, amplification or sequencing methods of the invention, and (viii) one or more hosts or host cells.

The invention also relates to compositions comprising the ligands (e.g., cationic compounds/molecules/compositions) of the invention bound to or complexed with one or more nucleic acid molecules as well as the ligand/nucleic acid molecule(s) complexes found in such compositions or made during the methods of the invention.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

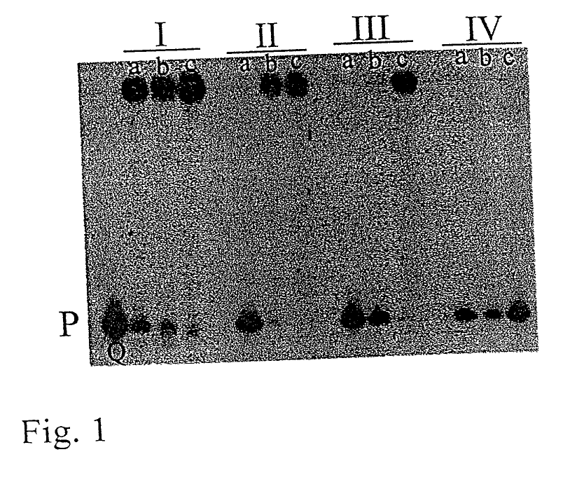

The polymerase activity of Tne DNA polymerase (D737A; 5′-3′ exonuclease deficient) was measured at ambient temperature, 37° C. and 72° C. in the presence and absence of the cationic composition Lipofectamine™ (available from Invitrogen Corporation, Life Technologies Division, Rockville, Md.). The DNA substrate used for the polymerase assay was a 34/60 mer primer/template. The 5′-terminus of the primer strand was labeled with 32P using T4 polynucleotide kinase. A polymerization reaction was initiated by the addition of Tne DNA polymerase to a solution of the DNA substrate in the presence of dNTP and MgCl₂. The reaction concentration of the DNA was about 10 nM, each of the four dNTP was 200 uM and MgCl₂ was 1.5 mM. LIPOFECTAMINE™ was added to the DNA-dNTP-Mg²⁺ solution and the mix was incubated for about 5 minutes at ambient temperature to allow the formation of DNA-cationic composition complex prior to the initiation of the reaction with Tne polymerase. For the control reaction (see FIG. 1; panel I), LIPOFECTAMINE™ was not present. The concentration of the Tne DNA polymerase was about 70 nM, whereas, the concentration of the LIPOFECTAMINE™ varied from 0 to 40 mM. The reactions were stopped at 4 minutes following addition of Tne.

The polymerase activity of Tne DNA polymerase was significantly inhibited at ambient temperature in the presence of 10 mM LIPOFECTAMINE™, whereas at 37° C. and 72° C. the reaction was not affected. The inhibition of the enzymatic activity is dependent to the concentration of the LIPOFECTAMINE™ under our experimental conditions. However, polymerization reaction is significantly inhibited even at 37° C. and 72° C. as the concentration of LIPOFECTAMINE™ is increased (see FIG. 1; panels III & IV).

### EXAMPLE 2

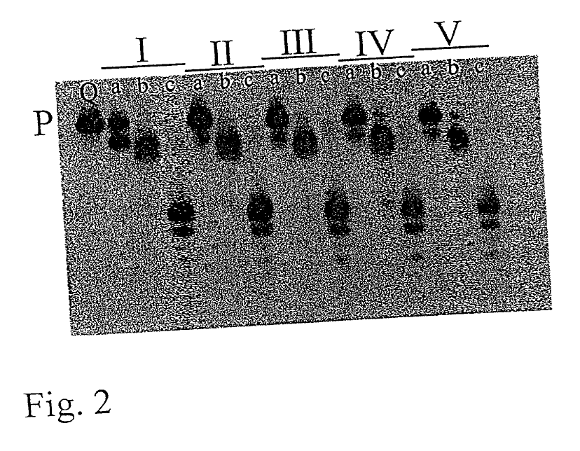

The 3′→5′ exo-nuclease activity of Tne DNA polymerase (5′-3′ exonuclease deficient) was measured using a single stranded 34-mer DNA substrate. The exo-nuclease directed DNA digestions were measured at ambient temperature, 37° C. and 72° C. in the presence and absence of the LIPOFECTAMINE™. The 5′-terminus of the oligonucleotide substrate was labeled with ³²P using T4 polynucleotide kinase. The exo-nuclease reaction was initiated by the addition of Tne DNA polymerase to a solution of the 34-mer oligonucleotide substrate in the presence of LIPOFECTAMINE™ and MgCl₂. LIPOFECTAMINE™ was added to the DNA solution and the mix was incubated for about 5 minutes at ambient temperature to allow the formation of DNA-cationic composition complex prior to the initiation of the exo-nuclease directed ssDNA digestion with Tne polymerase. For the control reaction (see FIG. 2; panel I), LIPOFECTAMINE™ was not present. For each reaction, the reaction concentration of DNA substrate was about 10 nM and the MgCl₂ was about 3 mM. The concentration of the Tne DNA polymerase was about 70 nM, whereas the concentration of the LIPOFECTAMINE™ varied from 0 to 60 mM.

The 3′→5′ exo-nuclease activity of Tne DNA polymerase was significantly inhibited at ambient temperature in the presence of the LIPOFECTAMINE™ under our experimental conditions. At 37° C. and 72° C., LIPOFECTAMINE™ was not a very effective inhibitor of the exo-nuclease activity of Tne even at 60 mM concentration of LIPOFECTAMINE™ (see FIG. 2, panel V). The above results suggest that LIPOFECTAMINE™ binds/protects ssDNA and dsDNA substrates with significantly different affinity.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A composition for inhibiting nucleic acid synthesis, comprising one or more cationic or polycationic molecules and/or compounds capable of binding or having affinity to one or more nucleic acid molecules.

2. The composition of claim 1, wherein said molecules or compounds are selected from the group consisting of histones, protamine, sperinine, spermidine, and high mobility group proteins.

3. The composition of claim 1, wherein said molecules or compounds are a synthetic or natural molecules or compounds.

4. The composition of claim 3, wherein said synthetic molecules or compounds are selected from the group consisting of polymers, amphiphilic aggregates, cationic lipids, and cationic liposome formulations.

5. The composition of claim 4, wherein said polymers are selected from the group consisting of DEAE-dextran, polybrene, polyhistidine, cationic polypeptide, and polylysine.

6. The compostion of claim 1, wherein said polymer is polylysine.

7. The composition of claim 4, wherein said amphiphilic aggregrates are selected from the group consisting of polyamidoamine cascade polymers, lipopolyamines, and polyethylenimine.

8. The composition of claim 4, wherein said cationic lipids or cationic liposome formulations are selected from the group consisting of “Transfectam™”, “DOTAP™”, “Ingene 6™”, “X-treme GENE Q2™”, “GeneJammer™”, “GenePorter™”, “Effectene™”, “Superfect™”, “LIPOFECTIN™”, “LIPOFECTACE™”, “LIPOFECTAMINE™”, “LIPOFECTAMINE 2000™”, “CELLFECTIN™”, and “DMRIE-C™”.

9. The composition of claim 8, wherein said cationic lipid or liposome formulation is “LipofectAMINE™.

10. The composition of claim 1, wherein said molecules or compounds are thermolabile.

11. The composition of claim 1, wherein said binding or affinity of said molecules or compounds are inhibited, reduced, substantially reduced, or eliminated under conditions for nucleic acid synthesis.

12. The composition of claim 1, wherein said molecules or compounds are dissociated or denatured or inactivated under conditions for nucleic acid synthesis.

13. The composition of claim 1, wherein said molecules or compounds are derived from a polypeptide.

14. The composition of claim 1, further comprising one or more enzymes having nucleic acid polymerase activity.

15. The composition of claim 14, wherein said enzyme is thermophilic.

16. The composition of claim 15, wherein said thermophilic enzyme maintains polymerase activity under conditions for nucleic acid synthesis.

17. The composition of claim 15, wherein said enzyme having nucleic acid polymerase activity is selected from the group consisting of a DNA polymerase, an RNA polymerase and a reverse transcriptase.

18. The composition of claim 17, wherein said DNA polymerase is selected from the group consisting of Taq, Tne, Tma, Pfu, VENT™, DEEPVENT™, KOD, and Tth DNA polymerases, and mutants, variants and derivatives thereof.

19. The composition of claim 17, wherein said reverse transcriptase is selected from the group consisting of M-MLV reverse transcriptase, RSV reverse transcriptase, AMV reverse transcriptase, RAV reverse transcriptase, MAV reverse transcriptase and HIV reverse transcriptase, and mutants, variants and derivatives thereof.

20. The composition of claim 17, wherein said reverse transcriptase is substantially reduced in RNase H activity.

21. A method for synthesizing a nucleic acid molecule, comprising:
mixing at least one nucleic acid template with one or more molecules or compounds of claim 1 to form a mixture; and
incubating said mixture under conditions sufficient to synthesize a first nucleic acid molecule complementary to all or a portion of said template.

22. The method according to claim 21, wherein said mixing is accomplished under conditions to prevent nucleic acid synthesis and/or to allow binding of said molecules or compounds to one or more nucleic acid synthesis substrates.

23. The method according to claim 21, wherein said synthesis of said first nucleic acid molecule is accomplished under conditions sufficient to dissociate or denature or inactivate said molecules or compounds and/or to inhibit, reduce, substantially reduce, or eliminate binding of said molecules or compounds to one or more nucleic acid synthesis substrates.

24. The method according to claim 21, wherein said synthesis is accomplished in the presence of at least one component selected from the group consisting of one or more nucleotides, one or more polypeptides having polymerase activity, and one or more primers.

25. The method according to claim 21, wherein said mixture comprises one or more nucleic acid molecules selected from the group consisting of a double-stranded nucleic acid template/primer complex, a single-stranded template and a single-stranded primer.

26. The method of claim 21, further comprising incubating said first nucleic acid molecule under conditions sufficient to make a second nucleic acid molecule complementary to all or a portion of said first nucleic acid molecule.

27. A nucleic acid molecule made according to the method of claim 21.

28. A method for amplifying a nucleic acid molecule comprising:
mixing at least one nucleic acid template with one or more of the molecules or compounds of claim 1; and
incubating said mixture under conditions sufficient to amplify a nucleic acid molecule complementary to all or a portion of said template.

29. The method according to claim 28, wherein said mixing is accomplished under conditions to prevent nucleic acid amplification and/or to allow binding of said molecules or compounds to one or more nucleic acid amplification substrates.

30. The method according to claim 28, wherein said amplifying is accomplished under conditions sufficient to dissociate or inactive or denature said molecules or compounds and/or to inhibit, reduce, substantially reduce, or eliminate binding of said molecules or compounds to one or more nucleic acid amplification substrates.

31. The method according to claim 28, wherein said amplifying is accomplished in the presence of at least one component selected from the group consisting of one or more nucleotides, one or more polypeptides having polymerase activity, and one or more primers.

32. The method according to claim 28, wherein said mixture comprises one or more nucleic acid molecules selected from the group consisting of double-stranded nucleic acid template/primer complex, a single-stranded template and a single-stranded primer.

33. A nucleic acid molecule amplified according to the method of claim 28.

34. A method for sequencing a nucleic acid molecule comprising:
mixing at least one nucleic acid molecule to be sequenced with one or more of the molecules or compounds of claim 1, and one or more terminating agents to form a mixture;
incubating said mixture under conditions sufficient to synthesize a population of molecules complementary to all or a portion of said molecule to be sequenced; and
separating said population to determine the nucleotide sequence of all or a portion of said molecule to be sequenced.

35. The method according to claim 34, wherein said mixing is accomplished under conditions sufficient to prevent synthesis and/or to allow binding of said molecules or compounds to one or more nucleic acid sequencing substrates.

36. The method according to claim 34, wherein said synthesis of a population of molecules complementary to all or a portion of said molecule to be sequenced is accomplished under conditions sufficient to dissociate or denature or inactivate said molecules or compounds and/or to inhibit, reduce, substantially reduce, or eliminate binding of said molecules or compounds to one or more nucleic acid sequencing substrates.

37. The method according to claim 34, wherein said synthesis is accomplished in the presence of at least one component selected from the group consisting of one or more nucleotides, one or more polypeptides having polymerase activity, and one or more primers.

38. The method according to claim 34, wherein said mixture comprises one or more nucleic acid molecules selected from the group consisting of a double-stranded molecule to be sequenced/primer complex, a single-stranded molecule to be sequenced, and a single-stranded primer.

39. A kit for use in synthesis of a nucleic acid molecule, said kit comprising one or more of the molecules or compounds of claim 1.

40. The kit of claim 39, further comprising one or more components selected from the group consisting of one or more nucleotides, one or more DNA polymerases, one or more reverse transcriptases, one or more suitable buffers, one or more primers and one or more terminating agents.

41. An inhibitory composition comprising one or more cationic or polycationic molecules or compounds having high affinity to nucleic acids.

42. A method of synthesizing a nucleic acid molecule comprising:
mixing at least one nucleic acid template with one or more molecules or compounds of claim 1 under conditions sufficient to prevent or inhibit nucleic acid synthesis; and
incubating said mixture under conditions sufficient to dissociate or denature or inactivate said cationic molecules or compounds sufficient to allow synthesis of a nucleic acid molecule complementary to all or a portion of said template.

43. A method of sequencing a DNA molecule, comprising:
(a) providing a first DNA molecule to be sequenced with one or more nucleotides, one or more molecules or compounds of claim 1, and at least one terminator nucleotide under conditions sufficient to prevent or inhibit nucleic acid synthesis;
(b) incubating the mixture of step (a) under conditions sufficient to dissociate or inactivate or denature said molecules or compounds sufficient to allow synthesis of a random population of DNA molecules complementary to said first DNA molecule, wherein said synthesized DNA molecules are shorter in length than said first DNA molecule and wherein said synthesized DNA molecules comprise a terminator nucleotide at their 5′ termini; and
(c) separating said synthesized DNA molecules by size so that at least a part of the nucleotide sequence of said first DNA molecule can be determined.

44. A method for amplifying a double-stranded DNA molecule, comprising:
(a) providing a first and second primer, wherein said first primer is complementary to a sequence at or near the 3′-termini of the first strand of said DNA molecule and said second primer is complementary to a sequence at or near the 3′-termini of the second strand of said DNA molecule and one or more molecules or compounds of claim 1, under conditions such that said molecules or compounds prevent or inhibit nucleic acid synthesis;
(b) incubating under conditions sufficient to dissociate or inactivate or denature said molecules or compounds sufficient to allow synthesis of a third DNA molecule complementary to said first strand and a fourth DNA molecule complementary to said second strand;
(c) denaturing said first and third strand, and said second and fourth strands; and
repeating steps (a) to (b) or (c) one or more times.

45. A method of preparing cDNA from mRNA, comprising
mixing one or more mRNA templates with one or more molecules or compounds of claim 1; and
incubating said mixture under conditions sufficient to synthesize a cDNA molecule complementary to all or a portion of said templates.

46. A method for amplifying a nucleic acid molecule comprising:
mixing at least one nucleic acid template with one or more molecules or compounds of claim 1 under conditions sufficient to prevent or inhibit nucleic acid amplification; and
incubating said mixture under conditions sufficient to dissociate or denature or inactivate said molecules or compounds sufficient to allow amplification of nucleic acid molecules complementary to all or a portion to said template.

47. A method to prevent or inhibit degradation of nucleic acid molecules comprising:
obtaining one or more nucleic acid ligands; and
contacting said ligands with one or more nucleic acid molecules under conditions sufficient to prevent or inhibit degradation of said nucleic molecules by one or more nucleases having nuclease activity.

48. The method of claim 47, wherein said ligands are polycationic or cationic molecules or compounds.

49. A composition for inhibiting nucleic acid synthesis comprising one or more cationic or polycationic molecules or compounds.

50. The composition of claim 49, wherein said molecules or compounds bind or have affinity to one or more nucleic acid molecules.

51. The composition of claim 49, further comprising at least one component selected from the group consisting of one or more nucleotides, one or more nucleic acid templates, one or more primers and one or more enzymes having nucleic acid polymerase activity.

52. A method to inhibit or prevent nucleic acid synthesis comprising:
mixing at least one nucleic acid template with one or more cationic or polycationic molecules or compounds; and
incubating said mixture under conditions sufficient to inhibit or prevent synthesis of a nucleic acid molecule complementary to all or a portion of said template.

53. The method of claim 52, wherein said mixture further comprises at least one component selected from the group consisting of one or more nucleotides, one or more nucleic acid templates, one or more primers and one or more enzymes having nucleic acid polymerase activity.

54. A method for introduction of one or more nucleic acid molecules in a host or host cell comprising:
synthesizing or amplifying one or more nucleic acid molecules in the presence of one or more nucleic acid ligands; and
introducing said synthesized or amplified nucleic acid molecules in one or more hosts or host cells in the presence of said ligands.

55. The method of claim 54, wherein at least one of said ligands is a cationic or polycationic compound or molecule.
